(19) United States
(12) Patent Application Publication
(10) Pub. No.: US 2024/0279733 A1
(43) Pub. Date: Aug. 22, 2024

(54) ORTHOGONAL HYBRIDIZATION

(71) Applicant: Illumina, Inc., San Diego, CA (US)

(72) Inventors: Fei Shen, San Diego, CA (US); Mathieu Lessard-Viger, San Diego, CA (US); Eric Brustad, San Diego, CA (US); Allison Meade, San Diego, CA (US); Esteban Armijo, San Diego, CA (US); Michael Howard, La Mesa, CA (US); Jeffrey Fisher, San Diego, CA (US); Jonathan Boutell, Bishops Stortford (GB); Ramon Saracho, San Diego, CA (US); Olivia Ghazinejad, San Diego, CA (US); Seth McDonald, San Diego, CA (US); Lena Storms, San Diego, CA (US); Jeffrey Brodin, San Diego, CA (US)

(73) Assignee: Illumina, Inc., San Diego, CA (US)

(21) Appl. No.: 18/567,989

(22) PCT Filed: Dec. 15, 2022
(86) PCT No.: PCT/US22/53002

§ 371 Date: Dec. 7, 2023

Related U.S. Application Data
(60) Provisional application No. 63/290,852, filed on Dec. 17, 2021.

(51) Int. Cl. C12Q 1/6874

(52) U.S. Cl. CPC C12Q 1/6874

(57) ABSTRACT

The present disclosure is directed to decoupling library capture (template seeding) from cluster generation to optimise both processes. This is achieved by introducing orthogonality between the seeding and clustering primer.

### CROSS-REFERENCE TO RELATED APPLICATION

This application claims the benefit of U.S. Provisional Patent Application No. 63/290,852, filed Dec. 17, 2021 and entitled “Orthogonal Hybridization,” the entire contents of which are incorporated by reference herein.

### FIELD

The present disclosure is generally directed to strategies for template capture and amplification during sequencing.

### BACKGROUND

The detection of analytes such as nucleic acid sequences that are present in a biological sample has been used as a method for identifying and classifying microorganisms, diagnosing infectious diseases, detecting and characterizing genetic abnormalities, identifying genetic changes associated with cancer, studying genetic susceptibility to disease, and measuring response to various types of treatment. A common technique for detecting analytes such as nucleic acid sequences in a biological sample is nucleic acid sequencing.

Advances in the study of biological molecules have been led, in part, by improvement in technologies used to characterise the molecules or their biological reactions. In particular, the study of the nucleic acids DNA and RNA has benefited from developing technologies used for sequence analysis.

Methods of nucleic acid amplification which allow amplification products to be immobilised on a solid support in order to form arrays comprised of clusters or “colonies” formed from a plurality of identical immobilised polynucleotide strands and a plurality of identical immobilised complementary strands are known. The nucleic acid molecules present in DNA colonies on the clustered arrays prepared according to these methods can provide templates for sequencing reactions.

One method for sequencing a polynucleotide template involves performing multiple extension reactions using a DNA polymerase to successively incorporate labelled nucleotides to a template strand. In such a “sequencing by synthesis” reaction a new nucleotide strand base-paired to the template strand is built up in the 5′ to 3′ direction by successive incorporation of individual nucleotides complementary to the template strand.

### SUMMARY

In one aspect of the disclosure, there is provided a solid support for use in sequencing comprising a plurality of capture moieties adapted to capture a template and a plurality of clustering primers; wherein the capture moieties are orthogonal to the clustering primers.

In another aspect of the disclosure, there is provided a nucleotide template library comprising a plurality of templates, wherein the templates comprise an insert and adaptor regions; wherein each adaptor region comprises a clustering primer and a complementary capture moiety, wherein the clustering primer and complementary capture moiety are orthogonal.

In a further aspect of the disclosure, there is provided an orthogonal capture fragment, comprising:
a first primer binding sequence substantially complementary to a primer binding sequence on a template (optionally wherein the first primer binding sequence is SEQ ID NO: 1 or variant thereof; or SEQ ID NO: 3 or variant thereof);
a complementary capture moiety, wherein said complementary capture moiety may optionally be complementary to the capture moiety as defined herein; and a linker between said first primer binding sequence and said complementary capture moiety, wherein the linker may optionally be a PEG linker;
   wherein said complementary capture moiety is orthogonal to said first primer binding sequence.

In another aspect of the disclosure, there is provided a method of sequencing a target nucleotide, wherein said method includes the step of preparing a double stranded library comprising templates as defined herein.

### BRIEF DESCRIPTION OF THE DRAWINGS

FIG. 1 shows a typical template for use in sequencing.

FIG. 2A shows quantified number of templates/nanowell obtained after clustering with different grafting primer inputs. FIG. 2B shows a simulated relation between grafting primer input and resulting templates/nanowell after different time of clustering. FIG. 2C shows relation between grafting primer input and resulting % PF (% Pass Filter mean).

FIG. 3 shows the relationship between primer density and sequencing intensity and % PF for standard P5/P7-based seeding and clustering.

FIG. 4 shows the relationship between primer density and sequencing intensity and % PF for decoupled seeding and clustering.

FIG. 5 shows an example of an orthogonal seeding strategy according to the present disclosure incorporating a PX′ oligo attached to a standard adaptor sequence via a linker.

FIG. 6A shows exemplary PCR library preparation. FIG. 6B shows a PCR-free library preparation. FIG. 6C shows further examples of PCR-free library preparation. FIG. 6D shows transposome-based library preparation steps according to the present disclosure. FIG. 6E shows a further library preparation.

FIG. 7 shows an exemplary non-nucleotide approach comprising biotin-bearing libraries able to hybridize to streptavidin grafted substrate surfaces.

FIG. 8 shows an exemplary non-nucleotide approach comprising click chemistry.

FIG. 9 shows an exemplary approach utilising dendrons.

FIGS. 10A-10B show schematics and models representing the underlying molecular events behind standard library seeding (FIG. 10A) and orthogonal seeding (FIG. 10B). A: Number of template binding sites in solution, S: Number of capture sites (primers) on surface.

FIG. 11 shows the robustness of a ds-library according to the present disclosure over a ss-library based on normalised first base intensity vs staging time at 35° C.

FIG. 12 shows PX-assisted double-stranded library seeding (P5/P7 grafting: 1.1 μM. PX grafting: 0.07 μM).

FIGS. 13A-13C shows a schematic representation of the data represented in FIG. 12.

FIG. 14 shows double-stranded seeding of orthogonal libraries being compatible with standard ExAmp-based template amplification.

FIG. 15 shows relation of PX surface density and library concentration on % occupancy, intensity and % PF.

FIG. 16 shows the effect of clustering primer (P5/P7) grafting input on % PF and C1 intensity in presence of orthogonal seeding capture (PX) motifs. Seeding concentration: 300 pM. PX: ˜39 per nanowell.

FIGS. 17 and 18 show the correlation between occupancy and seeding time at 40 and 50° C.

FIG. 19 shows the signal intensity of the system.

FIG. 20 shows % PF of occupied wells vs library and clustering primer input concentration variance to demonstrate impact on clonality.

FIG. 21 shows % PF and % occupancy at differing library concentrations for both standard and orthogonal hybridisation workflows.

FIG. 22 shows error rates at low and high clustering primer density.

FIGS. 23A-23B show an application of orthogonal seeding on multi-pad nanowells.

### DETAILED DESCRIPTION

The following features apply to all aspects of the present disclosure.

The present disclosure is directed to decoupling library capture (template seeding) from cluster generation to optimise both processes. This is achieved by introducing orthogonality between the seeding and clustering primers.

The present disclosure can be used in sequencing, for example pairwise sequencing. Methodology applicable to the present disclosure have been described in WO 08/041002, WO 07/052006, WO 98/44151, WO 00/18957, WO 02/06456, WO 07/107710, WO05/068656, U.S. Ser. No. 13/661,524 and US 2012/0316086, the contents of which are herein incorporated by reference. Further information can be found in US 20060024681, US 200602926U, WO 06110855, WO 06135342, WO 03074734, WO07010252, WO 07091077, WO 00179553 and WO 98/44152, the contents of which are herein incorporated by reference.

Sequencing generally comprises four fundamental steps: 1) library preparation to form a plurality of template molecules available for sequencing; 2) cluster generation to form an array of amplified single template molecules on a solid support; 3) sequencing the cluster array; and 4) data analysis to determine the target sequence.

Library preparation is the first step in any high-throughput sequencing platform. During library preparation, nucleic acid sequences, for example genomic DNA sample, or cDNA or RNA sample, is converted into a sequencing library, which can then be sequenced. By way of example with a DNA sample, the first step in library preparation is random fragmentation of the DNA sample. Sample DNA is first fragmented and the fragments of a specific size (typically 200-500 bp, but can be larger) are ligated, sub-cloned or “inserted” in-between two oligo adapters (adapter sequences). This may be followed by amplification and sequencing. The original sample DNA fragments are referred to as “inserts.” Alternatively, “tagmentation” can be used to attach the sample DNA to the adapters. In tagmentation, double-stranded DNA is simultaneously fragmented and tagged with adapter sequences and PCR primer binding sites. The combined reaction eliminates the need for a separate mechanical shearing step during library preparation. The target polynucleotides may advantageously also be size fractionated prior to modification with the adaptor sequences.

As used herein an “adapter” sequence comprises a short sequence-specific oligonucleotide that is ligated to the 5′ and 3′ ends of each DNA (or RNA) fragment in a sequencing library as part of library preparation. The adaptor sequence may further comprise non-peptide linkers.

As will be understood by the skilled person, a double-stranded nucleic acid will typically be formed from two complementary polynucleotide strands comprised of deoxyribonucleotides joined by phosphodiester bonds, but may additionally include one or more ribonucleotides and/or non-nucleotide chemical moieties and/or non-naturally occurring nucleotides and/or non-naturally occurring backbone linkages. In particular, the double-stranded nucleic acid may include non-nucleotide chemical moieties, e.g. linkers or spacers, at the 5′ end of one or both strands. By way of non-limiting example, the double-stranded nucleic acid may include methylated nucleotides, uracil bases, phosphorothioate groups, also peptide conjugates etc. Such non-DNA or non-natural modifications may be included in order to confer some desirable property to the nucleic acid, for example to enable covalent, non-covalent or metal-coordination attachment to a solid support, or to act as spacers to position the site of cleavage an optimal distance from the solid support. A single stranded nucleic acid consists of one such polynucleotide strand. Where a polynucleotide strand is only partially hybridised to a complementary strand—for example, a long polynucleotide strand hybridised to a short nucleotide primer—it may still be referred to herein as a single stranded nucleic acid.

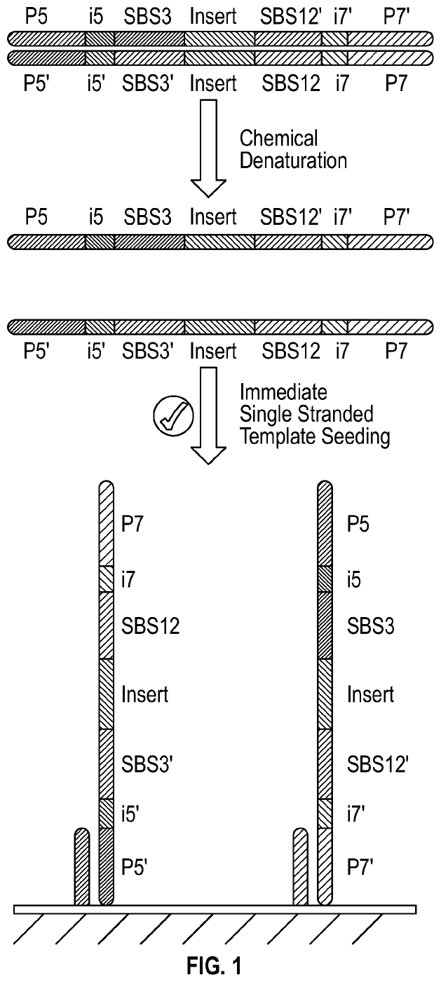

An example of a typical single-stranded nucleic acid template is shown in FIG. 1. In one embodiment, the template comprises, in the 5′ to 3′ direction, a first primer-binding sequence (e.g. P5), an index sequence (e.g. i5), a first sequencing binding site (e.g. SBS3), an insert, a second sequencing binding site (e.g. SBS12′), a second index sequence (e.g. i7′) and a second primer-binding sequence (e.g. P7′). In another embodiment, the template comprises, in the 3′ to 5′ direction, a first primer-binding site (e.g. P5′, which is complementary to P5), an index sequence (e.g. i5′, which is complementary to I5), a first sequencing binding site (e.g. SBS3′ which is complementary to SBS3), an insert, a second sequencing binding site (e.g. SBS12, which is complementary to SBS12), a second index sequence (e.g. i7, which is complementary to 17) and a second primer-binding sequence (e.g. P7, which is complementary to P7′). Either template is referred to herein as a “template strand” or “a single stranded template”. Both template strands annealed together as shown in FIG. 1, is referred to herein as “a double stranded template”. The combination of a primer-binding sequence, an index sequence and a sequencing binding site is referred to herein as an adaptor sequence, and a single insert is flanked by a 5′ adaptor sequence and a 3′ adaptor sequence.

The P5′ and P7′ primer-binding sequences are complementary to short primer sequences (or lawn primers) present on the surface of the flow cells. Binding of P5′ and P7′ to their complements (P5 and P7) on—for example—the surface of the flow cell, permits nucleic acid amplification. As used herein “′” denotes the complementary strand.

The primer-binding sequences in the adaptor which permit hybridisation to amplification primers will typically be around 20-40 nucleotides in length, although, in embodiments, the disclosure is not limited to sequences of this length. The precise identity of the amplification primers, and hence the cognate sequences in the adaptors, are generally not material to the disclosure, as long as the primer-binding sequences are able to interact with the amplification primers in order to direct PCR amplification. The sequence of the amplification primers may be specific for a particular target nucleic acid that it is desired to amplify, but in other embodiments these sequences may be “universal” primer sequences which enable amplification of any target nucleic acid of known or unknown sequence which has been modified to enable amplification with the universal primers. The criteria for design of PCR primers are generally well known to those of ordinary skill in the art. “Primer-binding sequences” may also be referred to as “clustering sequences” “clustering primers” or “cluster primers” in the present disclosure, and such terms may be used interchangeably.

The index sequences (also known as a barcode or tag sequence) are unique short DNA sequences that are added to each DNA fragment during library preparation. The unique sequences allow many libraries to be pooled together and sequenced simultaneously. Sequencing reads from pooled libraries are identified and sorted computationally, based on their barcodes, before final data analysis. Library multiplexing is also a useful technique when working with small genomes or targeting genomic regions of interest. Multiplexing with barcodes can exponentially increase the number of samples analysed in a single run, without drastically increasing run cost or run time. Examples of tag sequences are found in WO05068656, whose contents are incorporated herein by reference in their entirety. The tag can be read at the end of the first read by hybridizing an index read primer, or at the end of the second read, by using the surface primers as index read primers P7. The disclosure is not limited by the number of reads per cluster, for example two reads per cluster: three or more reads per cluster are obtainable simply by dehybridising a first extended sequencing primer, and rehybridising a second primer before or after a cluster repopulation/strand resynthesis step. Methods of preparing suitable samples for indexing are described in, for example U.S. 60/899,221. Single or dual indexing may also be used. With single indexing, up to 48 unique 6-base indexes can be used to generate up to 48 uniquely tagged libraries. With dual indexing, up to 24 unique 8-base Index 1 sequences and up to 16 unique 8-base Index 2 sequences can be used in combination to generate up to 384 uniquely tagged libraries. Pairs of indexes can also be used such that every i5 index and every i7 index are used only one time. With these unique dual indexes, it is possible to identify and filter indexed hopped reads, providing even higher confidence in multiplexed samples.

The sequencing binding sites are sequencing and/or index primer binding sites and indicates the starting point of the sequencing read. During the sequencing process, a sequencing primer anneals (i.e. hybridises) to a portion of the sequencing binding site on the template strand. The DNA polymerase enzyme binds to this site and incorporates complementary nucleotides base by base into the growing opposite strand. In one embodiment, the sequencing process comprises a first and second sequencing read. The first sequencing read may comprise the binding of a first sequencing primer (read 1 sequencing primer) to the first sequencing binding site (e.g. SBS3′) followed by synthesis and sequencing of the complementary strand. This leads to the sequencing of the insert. In a second step, an index sequencing primer (e.g. i7 sequencing primer) binds to a second sequencing binding site (e.g. SBS12) leading to synthesis and sequencing of the index sequence (e.g. sequencing of the i7 primer). The second sequencing read may comprise binding of an index sequencing primer (e.g. i5 sequencing primer) to the complement of the first sequencing binding site on the template (e.g. SBS3) and synthesis and sequencing of the index sequence (e.g. i5). In a second step, a second sequencing primer (read 2 sequencing primer) binds to the complement of the primer (e.g. i7 sequencing primer) binds to a second sequencing binding site (e.g. SBS12′) leading to synthesis and sequencing of the insert in the reverse direction.

Once a double stranded nucleic acid template library is formed, typically, the library will be subjected to denaturing conditions to provide single stranded nucleic acids. Suitable denaturing conditions will be apparent to the skilled reader with reference to standard molecular biology protocols (Sambrook et al., 2001, Molecular Cloning, A Laboratory Manual, 3rd Ed, Cold Spring Harbor Laboratory Press, Cold Spring Harbor Laboratory Press, NY; Current Protocols, eds Ausubel et al). In one embodiment, chemical denaturation, such as NaOH or formamide, is used. In another embodiment, the DNA is thermally denatured by heating.

Following denaturation, a single-stranded template library can be contacted in free solution onto a solid support comprising surface capture moieties (for example P5 and P7 primers). This solid support is typically a flowcell, although in alternative embodiments, seeding and clustering can be conducted off-flowcell using, for example, microbeads or the like.

As used herein, the term “solid support” refers to a rigid substrate that is insoluble in aqueous liquid. The substrate can be non-porous or porous. The substrate can optionally be capable of taking up a liquid (e.g. due to porosity) but will typically be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor, silica or silica-based materials including silicon and modified silicon, carbon, metals, inorganic glasses, optical fibre bundles, and polymers. A particularly useful material is glass. Other suitable substrate materials may include polymeric materials, plastics, silicon, quartz (fused silica), boro float glass, silica, silica-based materials, carbon, metals including gold, an optical fibre or optical fibre bundles, sapphire, or plastic materials such as COCs and epoxies. The particular material can be selected based on properties desired for a particular use. For example, materials that are transparent to a desired wavelength of radiation are useful for analytical techniques that will utilize radiation of the desired wavelength, such as one or more of the techniques set forth herein. Conversely, it may be desirable to select a material that does not pass radiation of a certain wavelength (e.g. being opaque, absorptive or reflective). This can be useful for formation of a mask to be used during manufacture of the structured substrate; or to be used for a chemical reaction or analytical detection carried out using the structured substrate. Other properties of a material that can be exploited are inertness or reactivity to certain reagents used in a downstream process; or ease of manipulation or low cost during a manufacturing process manufacture. Further examples of materials that can be used in the structured substrates or methods of the present disclosure are described in U.S. Ser. No. 13/661,524 and US Pat. App. Pub. No. 2012/0316086 A1, each of which is incorporated herein by reference.

The disclosure may make use of solid supports comprised of a substrate or matrix (e.g. glass slides, polymer beads etc) which has been “functionalised”, for example by application of a layer or coating of an intermediate material comprising reactive groups which permit covalent attachment to biomolecules, such as polynucleotides. Examples of such supports include, but are not limited to, a substrate such as glass. In such embodiments, the biomolecules (e.g. polynucleotides) may be directly covalently attached to the intermediate material but the intermediate material may itself be non-covalently attached to the substrate or matrix (e.g. the glass substrate). The term “covalent attachment to a solid support” is to be interpreted accordingly as encompassing this type of arrangement. Alternatively, the substrate such as glass may be treated to permit direct covalent attachment of a biomolecule; for example, glass may be treated with hydrochloric acid, thus exposing the hydroxyl groups of the glass, and phosphite-triester chemistry used to directly attach a nucleotide to the glass via a covalent bond between the hydroxyl group of the glass and the phosphate group of the nucleotide.

In other embodiments, the solid support may be “functionalised” by application of a layer or coating of an intermediate material comprising groups that permit non-covalent attachment to biomolecules. In such embodiments, the groups on the solid support may form one or more of ionic bonds, hydrogen bonds, hydrophobic interactions, π-π interactions, van der Waals interactions and host-guest interactions, to a corresponding group on the biomolecules (e.g. polynucleotides). The interactions formed between the group on the solid support and the corresponding group on the biomolecules may be configured to cause immobilisation or attachment under the conditions in which it is intended to use the support, for example in applications requiring nucleic acid amplification and/or sequencing. For example, the interactions formed between the group on the solid support and the corresponding group on the biomolecules may be configured such that the biomolecules remain attached to the solid support during amplification and/or sequencing.

In other embodiments, the solid support may be “functionalised” by application of an intermediate material comprising groups that permit attachment via metal-coordination bonds to biomolecules. In such embodiments, the groups on the solid support may include ligands (e.g. metal-coordination groups), which are able to bind with a metal moiety on the biomolecule. Alternatively, or in addition, the groups on the solid support may include metal moieties, which are able to bind with a ligand on the biomolecule. The metal-coordination interactions formed between the ligand and the metal moiety may be configured to cause immobilisation or attachment of the biomolecule under the conditions in which it is intended to use the support, for example in applications requiring nucleic acid amplification and/or sequencing. For example, the interactions formed between the group on the solid support and the corresponding group on the biomolecules may be configured such that the biomolecules remain attached to the solid support during amplification and/or sequencing.

When referring to immobilisation or attachment of molecules (e.g. nucleic acids) to a solid support, the terms “immobilised” and “attached” are used interchangeably herein and both terms are intended to encompass direct or indirect, covalent or non-covalent attachment, unless indicated otherwise, either explicitly or by context. In certain embodiments of the disclosure, covalent attachment may be preferred; in other embodiments, attachment using non-covalent interactions may be preferred; in yet other embodiments, attachment using metal-coordination bonds may be preferred. However, in general the molecules (e.g. nucleic acids) remain immobilised or attached to the support under the conditions in which it is intended to use the support, for example in applications requiring nucleic acid amplification and/or sequencing. When referring to attachment of nucleic acids to other nucleic acids, then the terms “immobilised” and “hybridised” are used herein, and generally refer to hydrogen bonding between complementary nucleic acids.

If the amplification is performed on beads, either with a single or multiple extendable primers, the beads may be analysed in solution, in individual wells of a microtitre or picotitre plate, immobilised in individual wells, for example in a fibre optic type device, or immobilised as an array on a solid support. The solid support may be a planar surface, for example a microscope slide, wherein the beads are deposited randomly and held in place with a film of polymer, for example agarose or acrylamide.

As described above, once a library comprising template nucleotide strands has been prepared, the templates are seeded onto a solid support and then amplified to generate a cluster of single template molecules.

By way of brief example, following attachment of the P5 and P7 primers, the solid support may be contacted with the template to be amplified under conditions which permit hybridisation (or annealing—such terms may be used interchangeably) between the template and the immobilised primers. The template is usually added in free solution under suitable hybridisation conditions, which will be apparent to the skilled reader. Typically, hybridisation conditions are, for example, 5×SSC at 40° C. Solid-phase amplification can then proceed. The first step of the amplification is a primer extension step in which nucleotides are added to the 3′ end of the immobilised primer using the template to produce a fully extended complementary strand. The template is then typically washed off the solid support. The complementary strand will include at its 3′ end a primer-binding sequence (i.e. either P5′ or P7′) which is capable of bridging to the second primer molecule immobilised on the solid support and binding. Further rounds of amplification (analogous to a standard PCR reaction) lead to the formation of clusters or colonies of template molecules bound to the solid support.

The present disclosure is directed to new library preparation, library capture (template seeding) and cluster generation techniques. The present disclosure enables the ability to decouple template seeding from cluster generation, and to optimise one or both processes. This is achieved by introducing orthogonality between seeding capture agents and clustering primers.

As outlined above, previous methodology utilises standard primers (P5/P7) grafted to the substrate surface to achieve both library capture (seeding) due to the presence of complementary sequences on the template (P5′ and P7′) and subsequent cluster generation. As such, the primers used for cluster generation are also used as the library template capture moiety. This interdependence of seeding and clustering complicates optimization of these processes.

In one embodiment, the sequence of the P5 primer-binding sequence comprises SEQ ID NO: 1 or a variant thereof, the sequence of the P5′ adaptor comprises SEQ ID NO: 3 or a variant thereof, the sequence of the P7 adaptor comprises SEQ ID NO: 2 or a variant thereof and the sequence of the P7′ adaptor comprises SEQ ID NO: 4 or a variant thereof.

In embodiments, the variant has at least 80% sequence identity to SEQ ID NO: 1, 2, 3 or 4. More preferably, the variant has at least 86%, 87%, 88%, 89%, 90%, 91%, 92%, 93%, 94%, 95%, 96%, 97%, 98%, or at least 99% overall sequence identity to SEQ ID NO: 1, 2, 3 or 4.

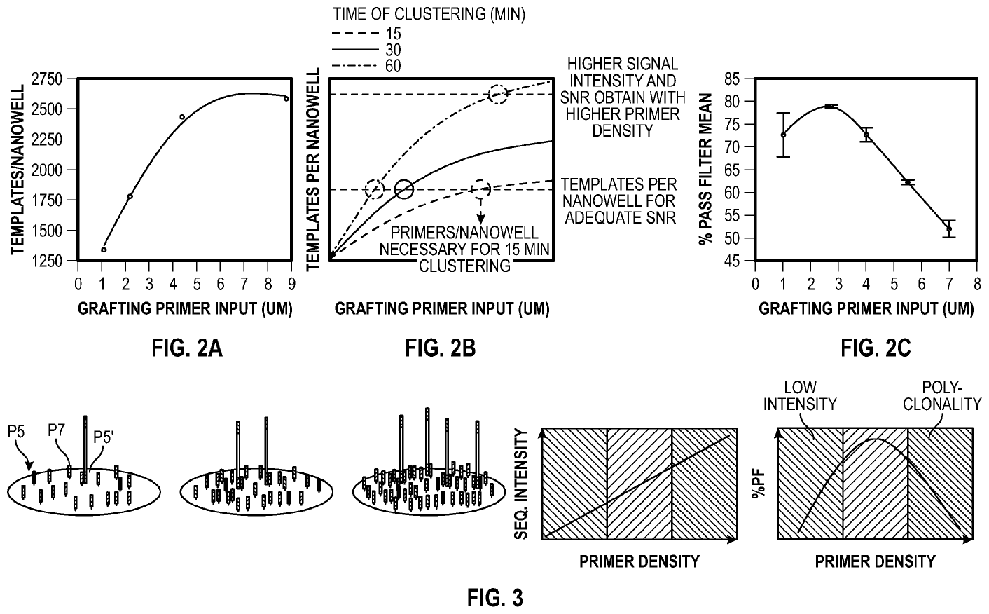

By way of example, it may be desirable to increase sequencing signal intensity (for example in response to lowering nanowell sizes). A strategy to achieve this is to increase primer density to promote amplification by maximising the number of amplified templates per nanowell. This is represented in FIG. 2A where the number of templates per nanowell is shown relative to grafting primer input. The impact of increased primer density is shown in FIG. 2B, which shows that increasing primer density can lead to a reduction in clustering time (a reduction in turnaround time or TAT). However, since grafting primers also serve as capture probes for template seeding, and hybridization kinetics are affected by the number of capture probes per nanowell, changing primer density impacts seeding efficiency. This is shown in FIG. 2C, where the % Pass Filter mean (% PF) is shown versus primer density. % PF is a measure of the ability of a nanowell to be successfully ‘read’ during sequencing. As the grafting density increases, there is an initial increase in % PF which is followed by a rapid decline, due to increased polyclonality within a well leading to a reduction in a clean readable target signal. Said another way, as the primer density increases, the likelihood of two or more templates hybridising onto the surface of the well increases. The presence of more than one template increases the likelihood of both templates being amplified leading to polyclonality and an increased likelihood that the signal strength is reduced or not readable. % PF can therefore be used to measure the degree of clonality. For the avoidance of doubt, while reference above is made to nanowells, the same concept is applicable to any solid support.

Thus, increasing primer density translates into an increase in the number of amplified templates, but also in the number of seeded molecules per nanowell. Consequently, as the nanowells become brighter, they also become more polyclonal. This is shown graphically in FIG. 3. The left hand image shows a representative flowcell surface comprising a plurality of primers (e.g. P5 and P7 primers). A single template (e.g. ss-DNA) is hybridised to a primer on the flowcell surface. Following a typical sequencing approach, the template is extended and then clustered using the free P5/P7 primers on the substrate surface to form a cluster of clonal (i.e. monoclonal) ss-DNA which can then be sequenced. It can be seen that the primer density increases as you move from left to right across the figure. During amplification, increased primer density leads to a larger cluster and an increase in sequence intensity. However, this also leads to an increase in the likelihood of multiple templates hybridising onto the surface. If two different templates hybridise and both form clusters within the nanowell, the nanowell will contain a mixture of different DNA samples, i.e. a single well will be polyclonal (as shown in the second and third representations). If the polyclonality is too high, (e.g. there is insufficient intensity from a single clonal family to provide a correct read during sequencing), then the read will be inconclusive or incorrect. This reduces the mean percentage % PF and therefore increases the number of reads which do not contain measurable data within the sequencing run. Since library hybridisation is at least in part a function of primer density, then as density increases in the flowcell the likelihood of multiple library seeding and polyclonality also increases.

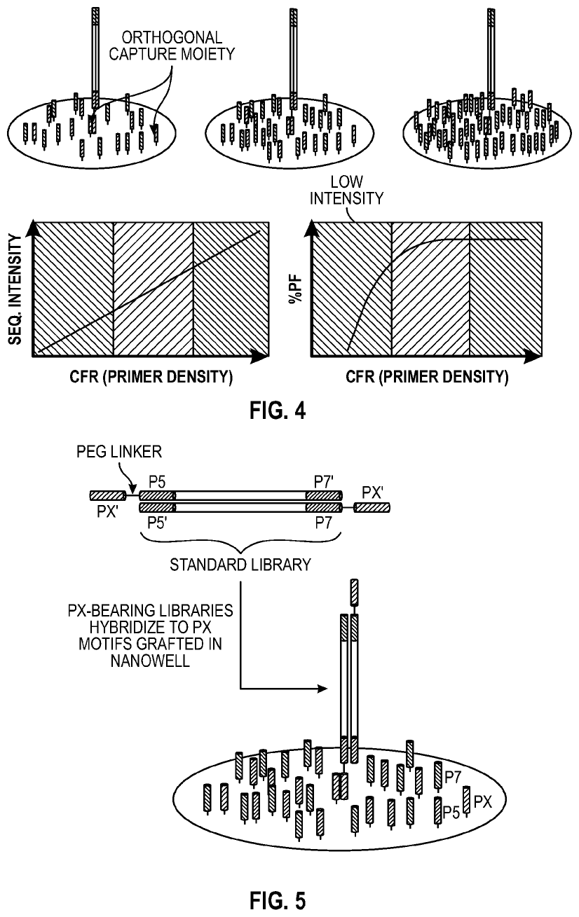

The present disclosure has identified a way to overcome these problems by removing the interdependence of seeding and clustering, allowing for both optimization of sequencing intensity and template library seeding. This is achieved by introducing orthogonality between the capture site used for seeding and the primers used for amplification. An example is shown graphically in FIG. 4. An orthogonal seeding capture moiety is used, which decouples seeding from clustering and repurposes P5/P7 as exclusive ‘clustering’ primers. By decoupling seeding from clustering, it is possible to increase clustering density (e.g. P5/P7) to maximize signal intensity, while keeping the seeding density constant to maintain optimal clonality.

By “orthogonal”, it is meant that the capture mechanism used to fix the template library to the flowcell surface is different from the primers used to generate the clusters. That is to say, the primers used during cluster generation are not also used as the capture moiety for the seeding step. These steps are instead decoupled such that the interdependence of seeding and clustering is removed.

Any suitable orthogonal capture mechanism can be used during seeding, provided the capture mechanism is orthogonal to the clustering primers. Non-limiting examples include a nucleotide based approach using an oligonucleotide sequence that is different to either of the clustering primers, or a non-nucleotide based binding approach, for example chemical capture such as biotin/streptavidin, click chemistry and the like.

### Nucleotide Binding

In an embodiment, an orthogonal oligonucleotide is used for seeding to capture the template (e.g. an orthogonal sequence or a seeding sequence). Such a seeding sequence on the flowcell surface may be designated PX, with the complementary sequence on the library template designated PX′. Any suitable seeding sequence may be used. An exemplary setup is shown in FIG. 5.

The present disclosure can be incorporated onto standard library templates. By way of example, a standard PCR-template is shown in FIG. 1. To the standard library is added a PX′, which is substantially complementary to the PX motif grafted to the substrate. A region is included between the orthogonal capture sequence (PX′) and the clustering sequences (P5/P7) that cannot be by-passed by DNA polymerases. An example of such a region is a PEG linker separating the PX′ sequence (seeding) from the clustering sequence (P5/P7). Commonly used PCR-based DNA polymerases cannot by-pass the PEG linker, terminating DNA polymerization before copying the PX′ sequence. Other linking strategies are possible to ensure PX′ is not extended. This allows for PX′ to remain single-stranded and available for hybridization at all times.

By “complementary” is meant that the blocking oligo has a sequence of nucleotides that can form a double-stranded structure by matching base-pairs with the adaptor or primer sequence or part thereof. By “substantially complementary” is meant that the blocking nucleotides has at least 85%, 90%, 95%, 98% or 99% or 100% overall sequence identical to the complementary sequence.

Exemplary spacers/linkers are identified below.

Thus, according to the present disclosure, genomic templates can be seeded as double-stranded DNA. This is in contrast to existing methodologies, which require denaturation of the dsDNA to form ssDNA for seeding. This difference is due to the P5/P7′ primers being within the dsDNA region, and therefore not accessible to P5/P7 surface primers during seeding.

In order to generate an orthogonal template library, PX′-primers comprising A-L-P may be used, where A represents the PX′ oligo, L represents a linker and P represents a sequence complementary to the primer binding sequence within the adaptor region (e.g. a sequence complementary to P5′ or P7′).

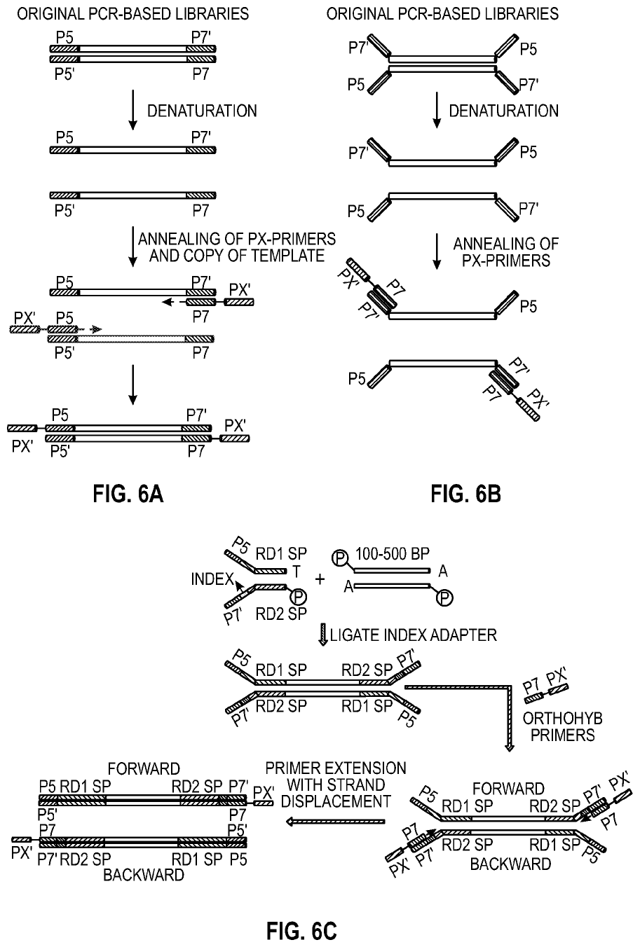

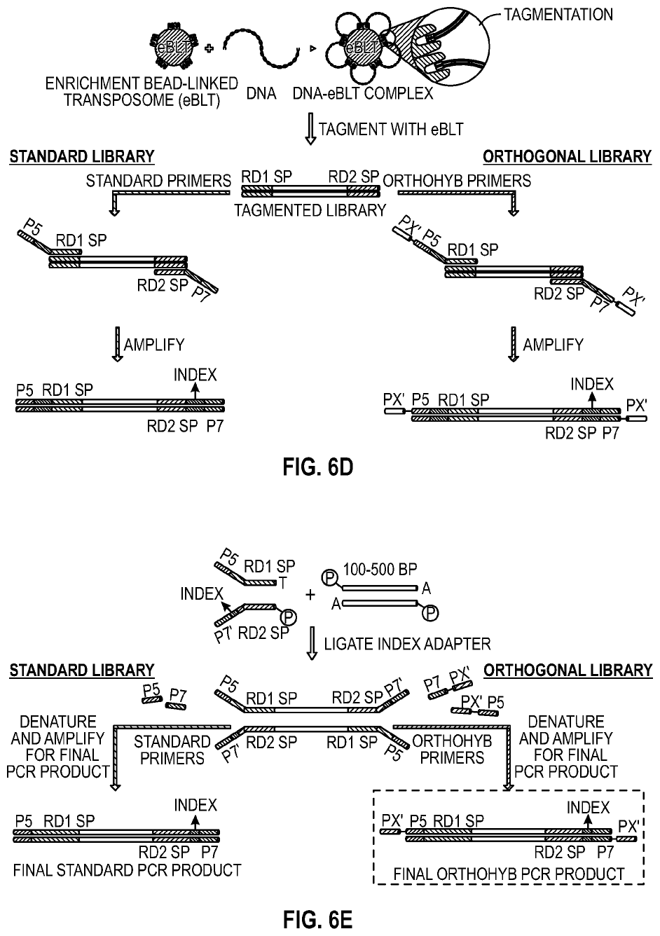

An exemplary PCR-based library preparation strategy according to the present disclosure is shown in FIG. 6A. In this example, a double-stranded template is prepared as described above, comprising fragmenting the library and ligating the adaptor sequence to the insert. This results in an insert sequence flanked at its 5′ and 3′ends by adaptor sequences comprising primer-binding sequences. Once the library is formed, the library is denatured and the orthogonal template (A-L-P) introduced during PCR enrichment. As shown in FIG. 6A, the complement of the primer-binding sequence, P binds (anneals) its complement (e.g. P5′ or P7′) in the template strand. Extension of the P7 or P5 primer leads to a double-stranded template with PX-L attached at the 5′ends. The denaturation, annealing and extension steps described above are known to the skilled person and can be carried out as summarised herein.

A different workflow is applied to PCR-free library preparation. An exemplary process is shown in FIG. 6B and FIG. 6C. In FIG. 6B, a PCR-free library is constructed by standard procedures and then denatured to produce free single stranded libraries. Upon neutralization of the denaturation reaction, a blocking oligo is added in excess. This oligo contains PX′-linker-sequence where the sequence is complementary to P7′ on the PCR-free 3′ termini. These blocking oligos affectively render P7′ double stranded so it cannot anneal to the FC, while at the same time providing PX′ for orthogonal hybridization. In FIG. 6C, the PCR-free library is not denatured. Instead, the same blocking oligo as above can be annealed to P7′ and then extended by a strand displacing polymerase to generate a double stranded library with orthogonal single stranded hybridization motif.

A different workflow is also used when tagmentation is used to attach the adaptor sequences. This is shown in FIG. 6D. In summary, the standard process for tagmenting adaptor sequences involves (a) integration of transposomes into genomic DNA to produce amplifiable and non-amplifiable library molecules, (b) cleaning of the library to remove transposase proteins and (c) annealing of adaptor sequences (each comprising the primer binding sites, index and sequencing-binding sites) and PCR amplification of the template. In the present workflow, the standard process is followed other than instead of using the standard adaptor sequences in step (c), adaptor sequences linked to Px are used as shown in the figure.

In another example of a PCR-library preparation strategy, the template library is fragmented to generate blunt-ends, and adenosine is added to the blunt ends of each strand to prepare the template for ligation to the adaptor sequences (each comprising the primer binding sites, index and sequencing-binding sites). Each adaptor sequence in this example will contain a thymine overhang on it's 3′ end providing a complementary overhang for ligating the adaptor sequence (which will bind to the adenosine on the template strand). The ligated insert sequence is then denatured and amplified using primer-binding sequences (e.g. P5 or P7) to produce the final double-stranded template library. This alternative standard process for generating a template library is shown in FIG. 6E. In the present workflow, the standard process is followed other than the use of the orthogonal template (A-L-P) replaces the use of primer-binding sequences (P5 or P7) to amplify the ligated template.

In all cases, complementary PX seeding sequences grafted to the substrate surface enable the library to be annealed to the substrate via PX/PX′ hybridisation.

Although not limiting, exemplary sequences are provided below by way of example, comprising PX′-seeding sequence for library preparation and a PX flowcell sequence for library capture:

SEQ ID NO: 5 PX′-P5:
(PX is underlined. P5 is bold)
5′ CCTCCTCCTCCTCCTCCTCCTCCT/iSp9/AATGATACGGCGACCA
CCGA 3′
SEQ ID NO: 6 PX′-P7:
(PX is underlined. P7 is bold)
5′ CCTCCTCCTCCTCCTCCTCCTCCT/iSp9/CAAGCAGAAGACGGCA
TAC 3′
PX substrate sequence:
SEQ ID NO: 7
5′ AGGAGGAGGAGGAGGAGGAGGAGG/iSp9/U-alkyne 3′

wherein iSp9 represents the following:

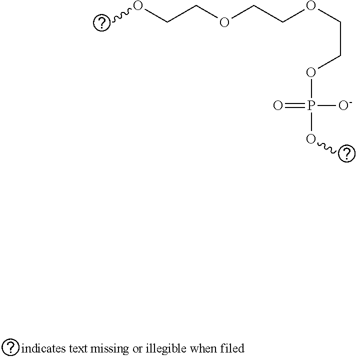

and wherein U-alkyne represents 5-ethynyluracil. Alternatively, the ethynyl group can be appended to the 5′-end of PX as immobilization via either orientation is functional.

While a single sequence may be selected for PX, the disclosure is not limited in this regard and any number of DNA sequences can be used as an orthogonal seeding sequence.

Further exemplary primers are shown below:

PX
SEQ ID NO: 8
AGGAGGAGGAGGAGGAGGAGGAGG
cPX (PX′)
SEQ ID NO: 9
CCTCCTCCTCCTCCTCCTCCTCCT
PA
SEQ ID NO: 10
GCTGGCACGTCCGAACGCTTCGTTAATCCGTTGAG
cPA (PA′)
SEQ ID NO: 11
CTCAACGGATTAACGAAGCGTTCGGACGTGCCAGC
PB
SEQ ID NO: 12
CGTCGTCTGCCATGGCGCTTCGGTGGATATGAACT
cPB (PB′)
SEQ ID NO: 13
AGTTCATATCCACCGAAGCGCCATGGCAGACGACG
PC
SEQ ID NO: 14
ACGGCCGCTAATATCAACGCGTCGAATCCGCAACT
cPV (PC′)
SEQ ID NO: 15
AGTTGCGGATTCGACGCGTTGATATTAGCGGCCGT
PD
SEQ ID NO: 16
GCCGCGTTACGTTAGCCGGACTATTCGATGCAGC
cPD (PD′)
SEQ ID NO: 17
GCTGCATCGAATAGTCCGGCTAACGTAACGCGGC

The above sequences are examples, but the present disclosure will work with any suitable orthogonal oligo strategy.

In embodiments, the present disclosure is directed to variants of the above sequences, wherein said variants have at least 50%, 55%, 60%, 65%, 70%, 75%, 80%, 85%, 90%, 95% or 99& sequence identity to SEQ ID NO: 5, 6, 7, 8, 9, 10, 11, 12, 13, 14, 15, 16, or 17.

### Non-Nucleotide Binding

In an embodiment, a non-nucleotide approach is used to capture the template. In one embodiment, the non-nucleotide approach is a chemical capture approach. The chemical capture approach may be configured to form non-covalent interactions, covalent bonds, or metal-coordination bonds with the template.

In some embodiments, the template may be attachable to the solid support by non-covalent interactions. These non-covalent interactions may include one or more of ionic bonds, hydrogen bonds, hydrophobic interactions, π-π interactions, van der Waals interactions and host-guest interactions. Where non-covalent interactions are used, the type of interaction is not particularly limited, provided that the interactions are (collectively) sufficiently strong for the template to remain attached to the solid support during extension. The non-covalent interactions may also be weak enough such that the template can then be removed from the solid support once a copy of the template has been extended on a surface primer.

As used herein, the term “ionic bond” refers to a chemical bond between two or more ions that involves an electrostatic attraction between a cation and an anion. For example, the cation may be selected from “metal cations”, as described herein, or “non-metal cations”. Non-metal cations may include ammonium salts (e.g. alkylammonium salts) or phosphonium salts (e.g. alkylphosphonium salts). The anion may be selected from phosphates, thiophosphates, phosphonates, thiophosphonates, phosphinates, thiophosphinates, sulfates, sulfonates, sulfites, sulfinates, carbonates, carboxylates, alkoxides, phenolates and thiophenolates.

As used herein, the term “hydrogen bond” refers to a bonding interaction between a lone pair on an electron-rich atom (e.g. nitrogen, oxygen or fluorine) and a hydrogen atom attached to an electronegative atom (e.g. nitrogen or oxygen).

As used herein, the term “host-guest interaction” refers to two or more groups which are able to form bound complexes via one or more types of non-covalent interactions by molecular recognition, such as ionic bonding, hydrogen bonding, hydrophobic interactions, van der Waals interactions and π-π interactions. For example, the host-guest interaction may include interactions formed between cucubiturils with adamantanes (e.g. 1-adamantylamine), ammonium ions (e.g. amino acids), ferrocenes; cyclodextrins with adamantanes (e.g. 1-adamantylamine), ammonium ions (e.g. amino acids), ferrocenes, calixarenes with adamantanes (e.g. 1-adamantylamine), ammonium ions (e.g. amino acids), ferrocenes; crown ethers (e.g. 18-crown-6, 15-crown-5, 12-crown-4) or cryptands (e.g. [2.2.2]cryptand) with cations (e.g. metal cations, ammonium ions); avidins (e.g. streptavidin) and biotin; and antibodies and haptens.

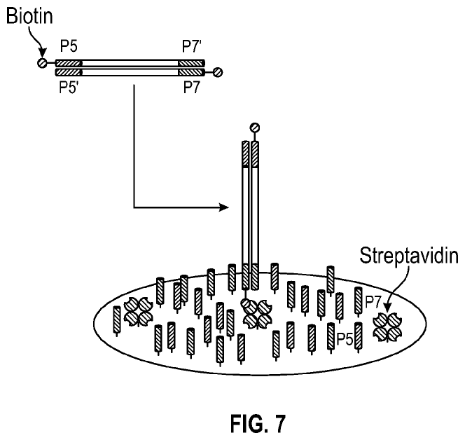

In a preferred embodiment, the non-covalent interaction is one formed between an avidin (e.g. streptavidin) and biotin. In some embodiments, both the solid support and the template may comprise biotin, and the template attached to the solid support via an avidin (e.g. streptavidin) bridging intermediary. In other embodiments, the solid support may comprise biotin, and attachable to an avidin (e.g. streptavidin) on the template. In other embodiments, the solid support may comprise an avidin (e.g. streptavidin), and attachable to a biotin moiety on the template. An example of this is shown in FIG. 7.

In other embodiments, the template may be attachable to the solid support by covalent bonds. Where covalent bonds are used, the bond may be stable such that the template remains attached to the solid support. Non-limiting examples of covalent bonds include alkylene linkages, alkenylene linkages, alkynylene linkages, ether linkages (e.g. ethylene glycol, propylene glycol, polyethylene glycol), amine linkages, ester linkages, amide linkages, carbocyclic or heterocyclic linkages, sulphur-based linkages (e.g. thioether, disulphide, polysulfide, or sulfoxide linkages), acetals, hemiaminal ethers, aminals, imines, hydrazones, boron-based linkages (e.g. boronic and borinic acids/esters), silicon-based linkages (e.g. silyl ether, siloxane), and phosphorus-based linkages (e.g. phosphite, phosphate).

In some embodiments, the covalent bond may be a reversible covalent bond such that the template can then be removed from the solid support once a copy of the template has been extended on a surface primer. In other embodiments, the covalent bond may be a non-reversible bond.

As used herein, the term “reversible covalent bond” refers to a covalent bond that can be cleaved for example under the application of heat, light or other (bio)chemical methods (e.g. by exposure to a degradation agent, such as an enzyme or a catalyst), while a “non-reversible covalent bond” is stable to degradation under such conditions. Non-limiting examples of reversible covalent bonds include thermally or photolytically cleavable cycloadducts (e.g. furan-maleimide cycloadducts), alkenylene linkages, esters, amides, acetals, hemiaminal ethers, aminals, imines, hydrazones, polysulfide linkages (e.g. disulfide linkages), boron-based linkages (e.g. boronic and borinic acids/esters), silicon-based linkages (e.g. silyl ether, siloxane), and phosphorus-based linkages (e.g. phosphite, phosphate)linkages.

In some embodiments, the solid support and/or the template may comprise a functional group selected from substituted or unsubstituted alkenyl, substituted or unsubstituted alkynyl, substituted or unsubstituted cycloalkenyl (e.g. norbornenyl, cis- or trans-cyclooctenyl), substituted or unsubstituted cycloalkynyl (e.g. cyclooctynyl, dibenzocyclooctynyl, bicyclononynyl), azido, substituted or unsubstituted tetrazinyl, substituted or unsubstituted hydrazonyl, substituted or unsubstituted tetrazolyl, aldehydes, ketones, carboxylic acids, sulfonyl fluorides, diazo (e.g. α-diazocarbonyl), substituted or unsubstituted oximes, hydroximoyl halides, nitrile oxide, nitrone, substituted or unsubstituted amino, substituted or unsubstituted hydrazines, thiol, or hydroxyl.

As used herein, the term “cycloadduct” refers to a cyclic structure formed from a cycloaddition reaction between two components (e.g. Diels-Alder or inverse electron demand Diels-Alder type cycloadditions between a diene and a dienophile, or 1,3-dipolar type cycloaddition between a dipole and a dipolarophile). The “cycloadduct” may be cleavable and undergo a retro-cycloaddition reaction to regenerate the two components (e.g. thermally or photolytically).

As used herein, the term “alkyl” or “alkylene” refers to monovalent or divalent straight and branched chain groups respectively having from 1 to 12 carbon atoms. Preferably, the alkyl or alkylene groups are straight or branched alkyl or alkylene groups having from 1 to 6 carbon atoms, more preferably straight or branched alkyl or alkylene groups having from 1 to 4 carbon atoms. An alkyl or alkylene group may comprise one or more “substituents”, as described herein.

As used herein, the term “alkenyl” or “alkenylene” refers to monovalent or divalent straight and branched chain groups respectively having from 1 to 12 carbon atoms, and which comprise at least one carbon-carbon double bond. Preferably, the alkenyl or alkenylene groups are straight or branched alkenyl or alkenylene groups having from 1 to 6 carbon atoms, more preferably straight or branched alkenyl or alkenylene groups having from 1 to 4 carbon atoms. An alkenyl or alkenylene group may comprise one or more “substituents”, as described herein.

As used herein, the term “alkynyl” or “alkynylene” refers to monovalent or divalent straight and branched chain groups respectively having from 1 to 12 carbon atoms, and which comprise at least one carbon-carbon triple bond. Preferably, the alkynyl or alkynylene groups are straight or branched alkynyl or alkynylene groups having from 1 to 6 carbon atoms, more preferably straight or branched alkynyl or alkynylene groups having from 1 to 4 carbon atoms. An alkynyl or alkynylene group may comprise one or more “substituents”, as described herein.

As used herein, the term “ether linkage” refers to a —O— group, where the oxygen atom is attached to two other carbon atoms at the points of attachment to the group.

As used herein, the term “amino” refers to a —N(R)(R′) group, where R and R′ are independently hydrogen or a “substituent” as defined herein. As used herein, the term “amine linkage” refers to a —NR— group, and where R is hydrogen or a “substituent” as defined herein.

As used herein, the term “ester linkage” refers to a —O—C(═O)— group, where the group is attached to two other carbon atoms at the points of attachment to the group.

As used herein, the term “amide linkage” refers to a —NR—C(═O)— group, where R is hydrogen or a “substituent” as described herein.

As used herein, the term “carbocyclic linkage” refers to a divalent “cycloalkylene” group, a divalent “cycloalkenylene” group, or a divalent “arylene” group.

A “cycloalkyl” or “cycloalkylene” group refers to an alkyl or alkylene group respectively comprising a closed ring comprising from 3 to 10 carbon atoms, for example, 3 to 6 carbon atoms. A cycloalkyl or cycloalkylene group may comprise one or more “substituents”, as described herein.

A “cycloalkenyl” or “cycloalkenylene” group refers to an alkenyl or alkenylene group respectively comprising a closed non-aromatic ring comprising from 3 to 10 carbon atoms, for example, 3 to 6 carbon atoms, and which contains at least one carbon-carbon double bond. A cycloalkenyl or cycloalkenylene group may comprise one or more “substituents”, as described herein.

A “cycloalkynyl” group refers to an alkynyl group respectively comprising a closed non-aromatic ring comprising from 8 to 12 carbon atoms, for example, 8 to 10 carbon atoms, and which contains at least one carbon-carbon triple bond. A cycloalkynyl group may comprise one or more “substituents”, as described herein.

An “aryl” or “arylene” group refers to a monovalent or divalent monocyclic, bicyclic or tricyclic aromatic group respectively containing from 6 to 14 carbon atoms in the ring. Common aryl groups include C₆-C₁₄ aryl or arylene, for example, C₆-C₁₀ aryl or arylene. An aryl or arylene group may comprise one or more “substituents”, as described herein.

As used herein, the term “heterocyclic linkage” refers to a divalent “heterocycloalkylene” group, or a divalent “heteroarylene” group.

A “heterocycloalkyl” or “heterocycloalkylene” group refers to a monovalent or divalent saturated or partially saturated 3 to 7 membered monocyclic, or 7 to 10 membered bicyclic ring system respectively, which consists of carbon atoms and from one to four heteroatoms independently selected from the group consisting of O, N, and S, wherein the nitrogen and sulfur heteroatoms may be optionally oxidised, the nitrogen may be optionally quaternised, and includes any bicyclic group in which any of the above-defined rings is fused to a benzene ring, and wherein the ring may be substituted on carbon or on a nitrogen atom if the resulting compound is stable. Non-limiting examples of “heterocycloalkyl” groups include pyrrolidinyl, tetrahydrofuranyl, dihydrofuranyl, tetrahydrothienyl, tetrahydrothiopyranyl, isoxazolinyl, piperidyl, morpholinyl, thiomorpholinyl, thioxanyl, piperazinyl, azetidinyl, oxetanyl, thietanyl, homopiperidyl, oxepanyl, thiepanyl, oxazepinyl, diazepinyl, thiazepinyl, 1,2,3,6-tetrahydropyridyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, dihydropyridazinyl (e.g. 1,4-dihydropyridazinyl), pyrazolidinyl, imidazolinyl, imidazolidinyl, 3-azabicyclo[3.1.0]hexyl, 3-azabicyclo[4.1.0]heptyl, 3H-indolyl, and quinolizinyl; non-limiting examples of “heterocycloalkylene” groups include the aforementioned groups in their divalent forms. A heterocycloalkyl or heterocycloalkylene group may comprise one or more “substituents”, as described herein.

A “heteroaryl” or “heteroarylene” group refers to monovalent or divalent aromatic groups having 5 to 14 ring atoms respectively (for example, 5 to 10 ring atoms) and containing carbon atoms and 1, 2 or 3 oxygen, nitrogen or sulfur heteroatoms. Non-limiting examples of “heteroaryl” groups include quinolyl including 8-quinolyl, isoquinolyl, coumarinyl including 8-coumarinyl, pyridyl, pyrazinyl, pyrazolyl, pyrimidinyl, pyridazinyl, furyl, pyrrolyl, thienyl, thiazolyl, isothiazolyl, triazolyl (e.g. 1,2,3-triazolyl), tetrazolyl, isoxazolyl, oxazolyl, imidazolyl, indolyl, isoindolyl, indazolyl, indolizinyl, phthalazinyl, pteridinyl, purinyl, oxadiazolyl, thiadiazolyl, furazanylene, pyridazinyl, triazinyl, cinnolinyl, benzimidazolyl, benzofuranyl, benzofurazanyl, benzothiophenyl, benzothiazolyl, benzoxazolyl, quinazolinyl, quinoxalinyl, naphthyridinyl and furopyridyl; non-limiting examples of “heteroarylene” groups include the aforementioned groups in their divalent forms. Where the heteroaryl (or heteroarylene) group contains a nitrogen atom in a ring, such nitrogen atom may be in the form of an N-oxide, e.g., a pyridyl N-oxide, pyrazinyl N-oxide, pyrimidinyl N-oxide and pyridazinyl N-oxide. A heteroaryl or heteroarylene group may comprise one or more “substituents”, as described herein.

As used herein, the term “sulfur-based linkage” refers to a —(S)ₙ— group, wherein n is 1 to 10, or 1 to 6. Preferably, n can be 1, forming a “sulfide” linkage; or n is 2 to 10 (e.g. 2 to 6), forming a “polysulfide” linkage. For example, n is 2, forming a “disulfide” linkage. In some embodiments, the sulfur atom may be optionally oxidised. In particular, a sulfur-based linkage may be a sulfone —S(═O)— linkage, or a sulfoxide —S(═O)₂— linkage.

As used herein, the term “acetal” refers to a —OC(R)(R′)O— group, where R and R′ are independently hydrogen or a “substituent” as described herein.

As used herein, the term “hemiaminal ether” refers to a —OC(R)(R′)NR″— group, where R, R′ and R″ are independently hydrogen or a “substituent” as described herein.

As used herein, the term “aminal” refers to a —NR(R′)(R″)NR′″— group, where R, R′, R″ and R′″ are independently hydrogen or a “substituent” as described herein.

As used herein, the term “imine” refers to a —C(R)═N— group, where R is hydrogen or a “substituent” as described herein.

As used herein, the term “hydrazone” refers to a —C(R)═N—NR′— group, where R and R′ are independently hydrogen or a “substituent” as described herein.

As used herein, the term “boron-based linkage” refers to a —(O)ₐ—B(OR)—(O)_{b}— group, where R is independently hydrogen or a “substituent” as described herein, and where a and b are independently 0 or 1.

As used herein, the term “silicon-based linkage” refers to a —(O)ₐ—Si(R)(R′)—(O)_{b}-group, where R and R′ are independently hydrogen or a “substituent” as described herein, and where a and b are independently 0 or 1.

As used herein, the term “phosphorus-based linkage” refers to a —(O)ₐ—P(R)—(O)_{b}-group, where R and R′ are independently hydrogen or a “substituent” as described herein, and where a and b are independently 0 or 1.

As used herein, the term “aldehyde” refers to a —C(═O)H group, where the group is attached to a carbon atom at the point of attachment to the group.

As used herein, the term “ketone” refers to a —C(═O)— group, where the group is attached to two other carbon atoms at the points of attachment to the group.

As used herein, the term “carboxylic acid” refers to a —C(═O)OH group.

As used herein, the term “sulfonyl fluoride” refers to a —S(═O)₂F group.

As used herein, the term “diazo” refers to a —C(═N⁺═N⁻)— group.

As used herein, the term “oxime” refers to a —C(R)═N—OR′ group, where R and R′ are independently hydrogen or a “substituent” as described herein.

As used herein, the term “hydroximoyl halide” refers to a —C(X)═N—OR group, where R is a hydrogen or a “substituent” as described herein, and X is a halogen.

As used herein, the term “nitrile oxide” refers to a —C≡N⁺—O⁻ group.

As used herein the term “nitrone” refers to a —C(═NR⁺—O⁻)— group, where R is a hydrogen or a “substituent” as described herein.

As used herein, the term “substituent” refers to groups such as OR′, ═O, SR′, SOR′, SO₂R′, NO₂, NHR′, NR′R′, ═N—R′, NHCOR′, N(COR′)₂, NHSO₂R′, NR′C(═NR′)NR′R′, CN, halogen, COR′, COOR′, OCOR′, OCONHR′, OCONR′R′, CONHR′, CONR′R′, protected OH, protected amino, protected SH, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted heterocycloalkyl, and substituted or unsubstituted heteroaryl, where each of the R′ groups is independently selected from the group consisting of hydrogen, OH, NO₂, NH₂, SH, CN, halogen, COH, COalkyl, CO₂H, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted heterocycloalkyl, and substituted or unsubstituted heteroaryl. Where such groups are themselves substituted, the substituents may be chosen from the foregoing list. In addition, where there are more than one R′ groups on a substituent, each R′ may be the same or different.

In other embodiments, the template may be attachable to the solid support by metal-coordination bonds. Where metal-coordination bonds are used, the bond may be strong enough such that the template remains attached to the solid support. The metal-coordination bond may be reversibly formed such that the template can then be removed from the solid support once a copy of the template has been extended on a surface primer.

As used herein, the term “metal-coordination bond” refers to an ionic bond and/or a dative covalent bond formed between a metal moiety and a ligand (e.g. a “metal-coordination group”, as described herein).

As used herein, the term “metal-coordination group” refers to a group which is able to coordinate with a metal moiety by forming an ionic bond and/or a dative covalent bond between the coordinating group and the metal moiety. Non-limiting examples of metal-coordination groups include benzenediols (e.g. catechols) or derivatives thereof; benzenetriols (e.g. gallols) or derivatives thereof, amino acids including histidine (e.g. polyhistidines such as His6 tag), serine, threonine, asparagine, glutamine, lysine, or cysteine; and ethylenediaminetetraacetic acid and derivatives thereof.

The ratio of metal-coordination group(s) to metal moieties can be tuned. There may be one, two or three coordinating groups per metal moiety.

As used herein, a “metal moiety” can be any metal moiety suitable to form ionic bonds, or to coordinate with a metal-coordinating group. For the metal-coordinating group, the metal moiety forms reversible ionic bonds and/or reversible dative covalent bonds with metal-coordination group(s). Suitable metal moieties include metal cations, metal oxides, metal hydroxides, metal carbides, metal nitrides and/or metal nanoparticles.

Particular metal cations include lithium, sodium, potassium, rubidium, caesium, beryllium, magnesium, calcium, strontium, barium, chromium, manganese, iron, cobalt, nickel, copper, silver, gold, platinum, palladium, zinc, cadmium, mercury, aluminium, gallium, indium, tin, lead and bismuth. Particularly preferred is nickel.

More particularly, suitable cations include alkali metal ions (e.g. Li⁺ lithium ion, Na⁺ sodium ion, K⁺ potassium ion, Rb⁺ rubidium ion, Cs⁺ caesium ion), alkaline earth metal ions (e.g. Be²⁺ beryllium ion, Mg²⁺ magnesium ion, Ca²⁺ calcium ion, Sr²⁺ strontium ion, Ba²⁺ barium ion), transition metal ions (e.g. Ti²⁺ titanium (II) ion, Ti⁴⁺ titanium (IV) ion, V²⁺ vanadium (II) ion, V³⁺ vanadium (III) ion, V⁴⁺ vanadium (IV) ion, V⁵⁺ vanadium (V) ion, Cr²⁺ chromium (II) ion, Cr³⁺ chromium (III) ion, Cr⁶⁺ chromium (VI) ion, Mn²⁺ manganese (II) ion, Mn³⁺ manganese (III) ion, Mn⁴⁺ manganese (IV) ion, Fe²⁺ iron (II) ion, Fe³⁺ iron (III) ion, Co²⁺ cobalt (II) ion, Co³⁺ cobalt (III) ion, Ni²⁺ nickel (II) ion, Ni³⁺ nickel (III) ion, Cu⁺ copper (I) ion, Cu²⁺ copper (II) ion, Ag⁺ silver ion, Au⁺ gold (I) ion, Au³⁺ gold (III) ion, Pt²⁺ platinum (II) ion, Pt⁴⁺ platinum (IV) ion, Pd²⁺ palladium (II) ion, Pd⁴⁺ palladium (IV) ion, Zn²⁺ zinc ion, Cd²⁺ cadmium ion, Hg⁺ mercury (I) ion, Hg²⁺ mercury (II) ion), Group III metal ions (e.g. Al³⁺ aluminium ion, Ga³⁺ gallium ion, In⁺ indium (I) ion, In³⁺ indium (III) ion), Group IV metal ions (e.g. Sn²⁺ tin (II) ion, Sn⁴⁺ tin (IV) ion, Pb²⁺ lead (II) ion, Pb⁴⁺ lead (IV) ion), and/or Group V metal ions (e.g. Bi³⁺ bismuth (III) ion, Bi⁵⁺ bismuth (V) ion). Ni²⁺ (II) ion is particularly preferred.

The metal moiety may be in the form of a metal salt. Suitable metal salts include but are not limited to halides, nitriles, hydroxides and the like.

The metal moiety may be in the form of an oxide or nanoparticle. For example, iron oxide nanoparticles may be used. Other suitable oxides or nanoparticles include iron oxides, iron nitrides, iron carbides, iron metal particles, nickel oxides, nickel carbides, nickel particles, titanium oxides, titanium metal particles, titanium nitrides, titanium carbides, silver metal particles and gold metal particles.

Preferably, the metal-coordination bond is one formed between nickel and histidine, such as nickel-His6 tag. The solid support may comprise nickel (e.g. nickel metal or nickel ions), and attachable to a histidine (e.g. His6 tag) moiety on the biomolecule. Alternatively, the solid support may comprise a histidine (e.g. His6 tag), and attachable to nickel (e.g. nickel metal or nickel ions) on the template.

In an embodiment, the template is captured on the surface of the flowcell by a chemical interaction on the flow cell surface. The flowcell may comprise a functionalized polymer coating layer which can be utilised to achieve chemical capture. The functionalized polymer coating layer may include one or more functional groups selected from substituted or unsubstituted alkenyl, substituted or unsubstituted alkynyl, substituted or unsubstituted cycloalkenyl (e.g. norbornenyl, cis- or trans-cyclooctenyl), substituted or unsubstituted cycloalkynyl (e.g. cyclooctynyl, dibenzocyclooctynyl, bicyclononynyl), azido, substituted or unsubstituted tetrazinyl, substituted or unsubstituted hydrazonyl, substituted or unsubstituted tetrazolyl, aldehydes, ketones, carboxylic acids, sulfonyl fluorides, diazo (e.g. α-diazocarbonyl), substituted or unsubstituted oximes, hydroximoyl halides, nitrile oxide, nitrone, substituted or unsubstituted amino, substituted or unsubstituted hydrazines, thiol, or hydroxyl. One example of a functionalized polymer coating layer is poly(N-(5-azidoacetamidylpentyl)acrylamide-co-acrylamide (PAZAM).

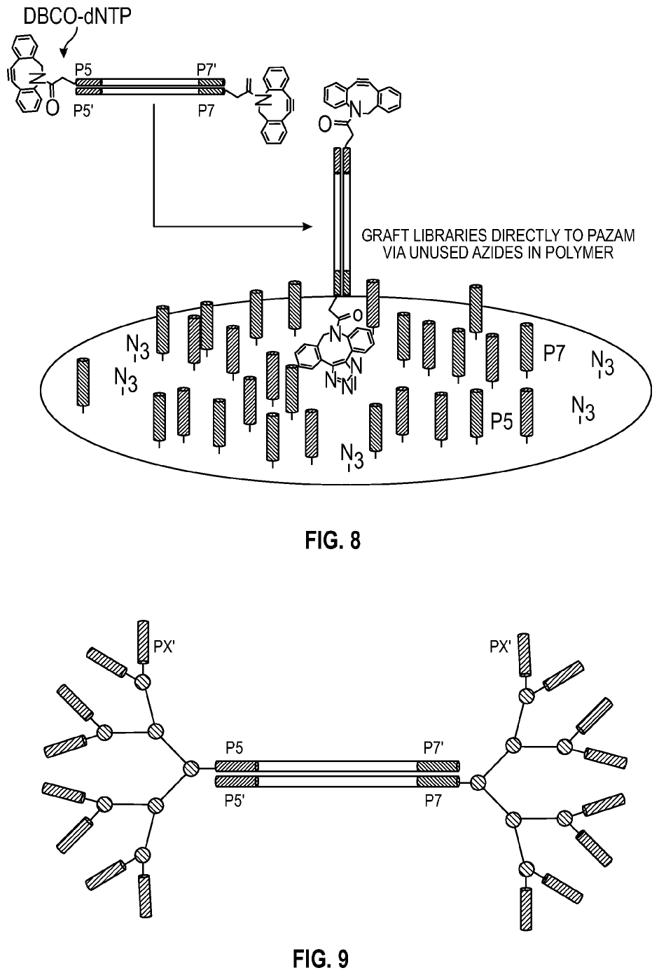

In an embodiment, the non-nucleotide approach comprises functional groups configured to form linkages by click chemistry. Such linkages may include linkages formed using thiol-ene click chemistry (e.g. between thiols and alkenyl reactive groups), copper-catalysed azide-alkyne cycloaddition (e.g. between azides and alkynyl reactive groups), strain-promoted dipolar cycloaddition (e.g. between azides, nitrile oxides or nitrones with cycloalkenyl/cycloalkynyl reactive groups; nitrile oxides may, for example, be generated in situ from oximes and hydroximoyl halides), strain-promoted Diels-Alder reactions (e.g. between tetrazines and cycloalkenyl/cycloalkynyl reactive groups), alkene-tetrazole photoclick reactions (e.g. between alkenyl and tetrazole reactive groups), and SuFEx click chemistry (e.g. between sulfonyl fluorides and nucleophiles such as carboxylic acids, thiols, hydroxyl and amino reactive groups). An exemplary click chemistry approach is shown in FIG. 8 comprising libraries containing DBCO-dNTPs on P5 and P7 ends covalently binding to unused azides present on a flowcell surface (for example within a PAZAM coating).

By way of further example, the capture motifs could be attached to the library using dendrons. An example of dendron-assisted seeding via PX motifs is shown in FIG. 9, which shows PX′-dendrons libraries, which can hybridize to PX motifs grafted on nanowells. The large number of PX′ motifs per library may improve seeding kinetics and/or efficiency.

### Linker

A spacer or linker may be provided between the capture moiety and the adaptor. For example, the spacer or linker may be provided between the capture moiety from the clustering sequence (P5/P7).

The linker may be a carbon-containing chain with a formula (CH2)n wherein “n” is from 1 to about 1500, for example less than about 1000, preferably less than 100, e.g. from 2-50, particularly 5-25.

Linkers which do not consist of only carbon atoms may also be used. Such linkers may include polyethylene glycol (PEG).

A particular linker is iSp9 (Spacer 9) which is a triethylene glycol spacer that can be incorporated at the 5′-end or 3-end of an oligo or internally.

Linkers formed primarily from chains of carbon atoms and from PEG may be modified so as to contain functional groups which interrupt the chains. Examples of such groups include ketones, esters, amines, amides, ethers, thioethers, sulfoxides, sulfones. Separately or in combination with the presence of such functional groups may be employed alkene, alkyne, aromatic or heteroaromatic moieties, or cyclic aliphatic moieties (e.g. cyclohexyl). Cyclohexyl or phenyl rings may, for example, be connected to a PEG or (CH₂)n chain through their 1- and 4-positions.

As an alternative to the linkers described above, which are primarily based on linear chains of saturated carbon atoms, optionally interrupted with unsaturated carbon atoms or heteroatoms, other linkers may be envisaged which are based on nucleic acids or monosaccharide units (e.g. dextrose). It is also within the scope of this disclosure to utilise peptides as linkers.

A variety of other linkers may be employed. The linker should be stable under conditions under which the polynucleotides are intended to be used subsequently, e.g. conditions used in DNA amplification. The linked should also be such that it is not by-passed by DNA polymerases, terminating DNA polymerization before copying the capture moiety sequence (if it is nucleotide based such as a PX′ sequence). This allows for PX′ to remain single-stranded and available for hybridization at all times.

The above embodiments of nucleotide and non-nucleotide capture moieties and linkers are not intended to be limited and merely provide examples of orthogonal strategies that can be used with the present disclosure.

Decoupling the capture agent from the clustering primers leads to a number of improvements on current processes.

Decoupling the capture agent from the clustering primer enables the template library to be seeded as double stranded DNA (dsDNA). Double-stranded seeding eliminates the need for library denaturation, which improves overall turnaround time.

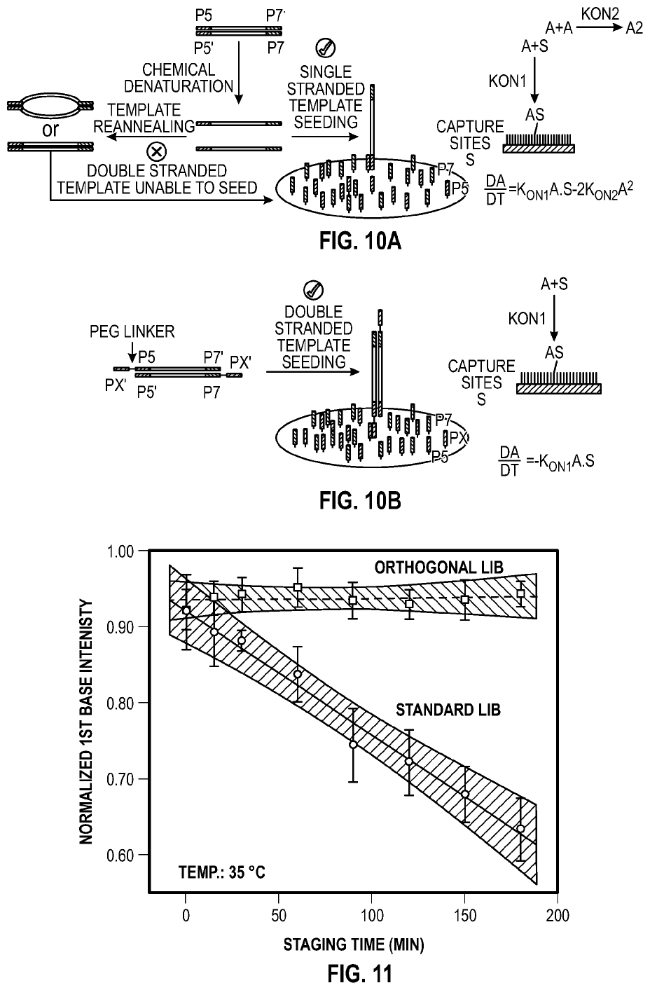

The ability to use dsDNA has further advantages that are shown in FIGS. 10A-10B. For the avoidance of doubt, although FIGS. 10A-10B show PX as the orthogonal capture moiety, non-nucleotide based approaches as defined herein equally achieve the same advantage. FIGS. 10A-10B demonstrate kinetic modeling best to describe PCR-amplified library seeding as a competition between surface hybridization and library reannealing in solution. FIG. 10A shows that single strand seeding of particularly PCR-amplified libraries requires the denatured library be kept cool and loaded quickly onto the flow cell in order to minimize reannealing which adversely impacts seeding. Denatured libraries stored for longer periods of time can reanneal, especially at the complementary adaptor ends. Reannealed strands cannot hybridize to surface primers in standard methodology. In addition to lowering the overall seeding efficiency, the time-dependent stability of the single stranded library can greatly impact seeding robustness and reproducibility, which in turns can affect sequencing quality.

In contrast, decoupling the capture agent from the clustering primers allows for the seeding of double stranded templates which eliminates competitive rehybridization since the availability of the PX′ motifs do not change with temperature and time. This can improve seeding efficiency and reproducibility. This can also influence the template concentration required for seeding.

The ability of the present disclosure to overcome seeding efficiency and reproducibility has been demonstrated in Example 1 and FIG. 11. It can be seen that the ss-library demonstrated a linear reduction in effectiveness due to re-annealing of the ss-templates. Non-productive template reannealing is both temperature dependent and concentration dependent. It decreases seeding efficiency and slows down seeding kinetics. For these reasons, denatured libraries need to be loaded quickly onto the flow cell to remain sufficiently single-stranded to enable efficient seeding. Delays in library loading adversely affects ultimate occupancy and flowcell yield. In contrast, the dsDNA library was both time and temperature independent.

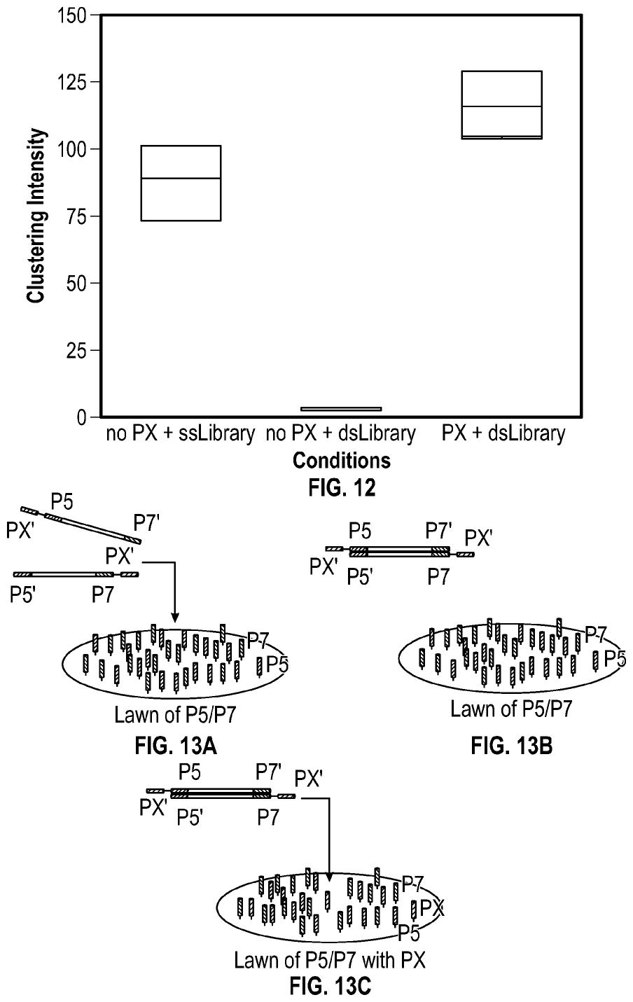

The ability for orthogonal dsDNA seeding libraries and decoupled capture agents to both seed and cluster is demonstrated in Example 2 and show in FIGS. 12 and 13. FIG. 12 demonstrates that a traditional ss-library with no orthogonal seeding has a high rate of clustering intensity. In contrast, if a ds-library with orthogonal seeding primer is used on a flowcell without a complementary capture agent, then no clustering intensity is seen meaning there was no template capture. However, an orthogonal capture strategy according to the present disclosure is shows high clustering intensity demonstrating that the present disclosure was able to both achieve capture and clustering.

This is conceptually shown in FIGS. 13A-13C. FIG. 13A shows denatured orthogonal libraries being captured on a standard flowcell without the need for orthogonal capture agents on the flowcell surface. FIG. 13B shows that ds-libraries cannot be captured on a flowcell which does not have corresponding orthogonal capture agents. FIG. 13C shows that ds-libraries can be captured in presence of a corresponding orthogonal capture agents. Although not shown, denatured ss-libraries can also be captured on a flowcell comprising orthogonal capture agents via the standard clustering primers. As such, flowcells according to the present disclosure can be back-compatible with ss-libraries (albeit taking into account any polyclonality disadvantages due to primer density) and ds-libraries according to the present disclosure can be denatured and used in a standard flowcell.

A further advantage of the present disclosure is that clustering primer density can be adjusted to achieve a desired signal intensity, for example based on nanowell size, target density and the detection system used (for example CMOS vs optical). Also, capturing agent density can be separately adjusted to obtain optimal (mono)clonality.

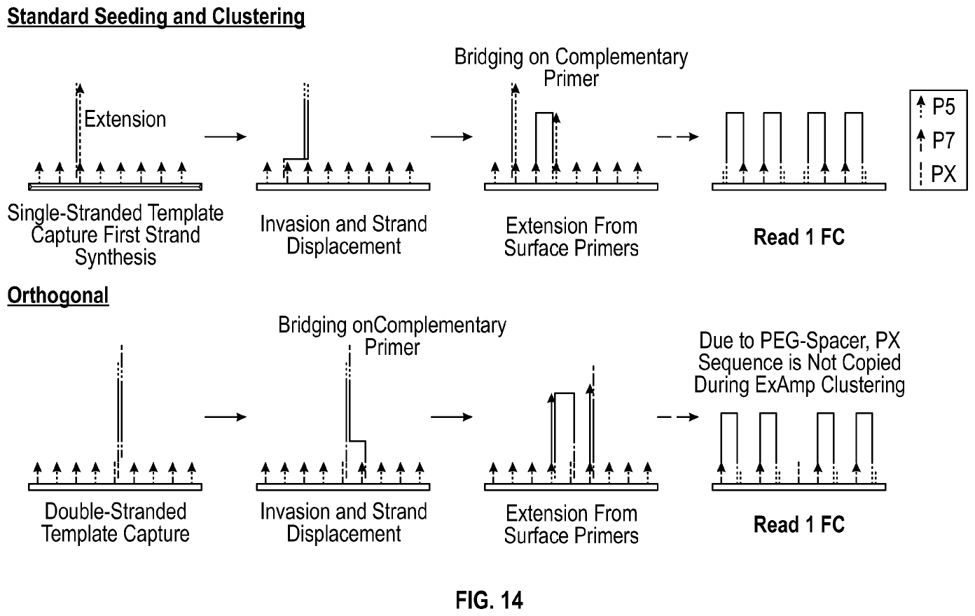

FIG. 14 shows an example of the ability for templates made according to the present disclosure to cluster. The top row shows a standard process involving template capture followed by strand synthesis. Invasion to an adjacent primer followed by strand displacement can then occur. The strands can then extend by bridging onto a complementary primer leading to cluster amplification and finally first read sequencing. According to an orthogonal approach from the present disclosure, the library template is captured as a ds-template on the orthogonal capture moiety. There immediately follows invasion and strand displacement. It can be noted that the first strand synthesis step is not required since the template is already double stranded. After displacement, the original template strand may bridge onto a complementary primer. Also, the displaced strand can be extended. It is noted that the orthogonal moiety on the template strands (in the example shown a PX sequence) is not copied during clustering. The strands are thereafter extended via cluster amplification and sequenced in the usual way.

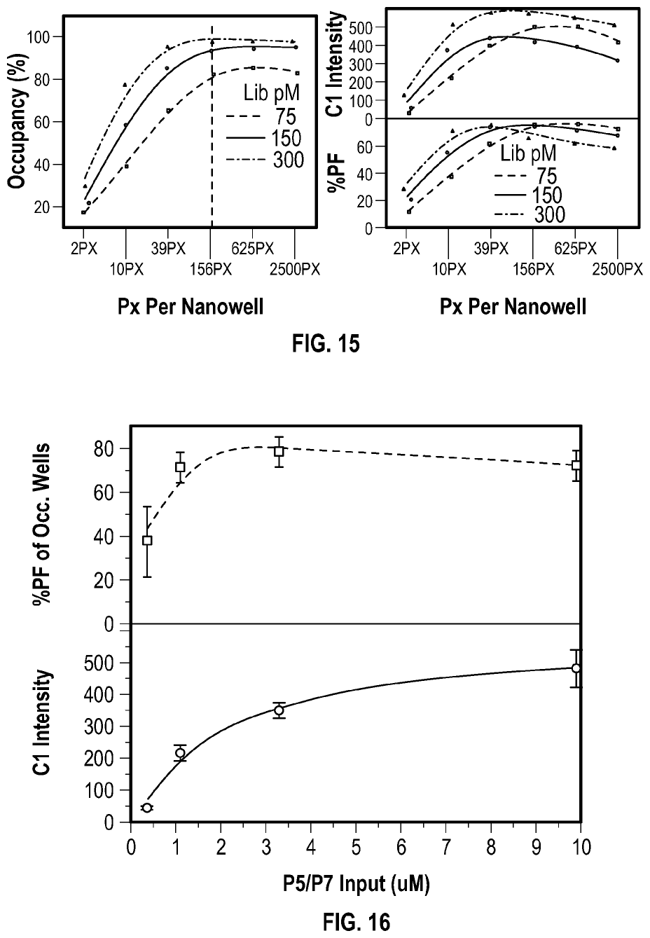

The relationship between capture agent surface density and library concentration was evaluated in Example 3 and FIG. 15 where it can be seen that capture agent surface density and/or library concentration can be optimised to maximise intensity and % PF. It can be seen, for example, when working with a 300 pM library, that having 39 capture probes (PX) is optimised versus a significantly higher number being present in traditional flowcells.

In the present disclosure, a discrete area of the solid support is intended to comprise a clonal cluster which is then sequenced to determine the sequence of the insert DNA replicated within the clonal cluster. This area may traditionally be a nanowell on a flowcell, but in alternative embodiments may be a microbead or other discrete area.

In an embodiment individual nanowells (or other discrete areas) comprises, on average, between around 1 to 5000 capture moieties. In a preferred embodiment, there are, on average, between around 1 to 2500, 1 to 1000, 1 to 625, 1 to 500, 1 to 300, 1 to 200, 1 to 156, 1 to 100, 1 to 80, 10 to 80, 1 to 60, 20 to 60, 30 to 50, 1 to 50, or around 35 to 45, or around 35, 36, 37, 38, 39, 40, 41, 42, 43, 43 or 45 capture moieties per individual nanowell (or other discrete area). The capture moieties are usually between present at a ratio of between 1:100 and 1:10 to the clustering primers. Average may be mean or median. Preferably average is mean. Average density can be calculated by fluorescently tagging all the available primers on a primed flow cell surface, and creating a standard curve between known concentration and fluorescence, to which the fluorescence of an individual nanowell can then be compared.

In a preferred embodiment, the library is seeded at 75 pM and there are, on average, between around 50 to 5000, 50 to 2500, 50 to 1000, 50 to 625, 50 to 500, 50 to 300, 50 to 200, 100 to 180, 120 to 170, 140 to 170, or around 150 to 160; or around 150, 151, 152, 153, 154, 155, 156, 157, 158, 159, or 160 capture moieties per individual nanowell (or other discrete area).

In a preferred embodiment, the library is seeded at 150 pM and there are, on average, between around 10 to 5000, 10 to 2500, 10 to 1000, 10 to 625, 10 to 500, 10 to 300, 10 to 200, 10 to 200, 10 to 150, or around 10 to 120 capture moieties per individual nanowell (or other discrete area).

In a preferred embodiment, the library is seeded at 300 pM and there are, on average, between around 1 to 5000, 1 to 2500, 1 to 1000, 1 to 625, 1 to 500, 1 to 300, 1 to 200, 1 to 156, 1 to 100, 1 to 80, 10 to 80, 1 to 60, 20 to 60, 30 to 50, 1 to 50, or around 35 to 45, or around 35, 36, 37, 38, 39, 40, 41, 42, 43, 43 or 45 capture moieties per individual nanowell (or other discrete area).

Other library concentration and capture moiety densities are envisaged and encompassed by the present disclosure.

Decoupling seeding from clustering also enables optimisation of sequencing intensity (based on higher clustering primer densities) whilst maintaining workable clonality. This is demonstrated in Example 4 and FIG. 16, which demonstrates increased C1 intensity without a significant reduction in % PF.

In an embodiment individual nanowells (or other discrete areas) comprises, on average, between 10,000 and 30,000 clustering primers. In a further embodiment, individual nanowells (or other discrete areas) comprise, on average, above 5000, 6000, 7000, 8000, 9000, 10,000, 11,000, 12,000, 13,000, 14,000, 15,000, 16,000, 17,000, 18,000, 19,000 20,000, 25,000, 30,000, 35,000, 40,000, 45,000 or 50,000 clustering primers. In some instances individual nanowells may comprise as many as 100, 000 clustering primers. Average may be mean or median. Preferably average is mean. Average density can be calculated by measuring fluorescence intensity and comparing it to a standard curve.

In an embodiment, the ratio of capture moieties: clustering primer on an individual nanowell (or other discrete area) is about 1:2, 1:3, 1:4, 1:5, 1:6, 1:7, 1:8, 1:9, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, 1:110, 1:120, 1:130, 1:140, 1:150, 1:160, 1:170, 1:180, 1:190, 1:200, 1:220, 1:240, 1:260, 1:280, 1:300, 1:320, 1:340, 1:360, 1:380, 1:400, 1:420, 1:440, 1:460, 1:480, or 1:500 about 1:1000 or more. Preferred ratios are between 1:10 and 1:1000.

In an embodiment, the ratio of capture moieties: clustering primer on an individual nanowell (or other discrete area) is about 1:>2, 1:>3, 1:>4, 1:>5, 1:>6, 1:>7, 1:>8, 1:>9, 1:>10, 1:>15, 1:>20, 1:>25, 1:>30, 1:>35, 1:>40, 1:>45, 1:>50, 1:>55, 1:>60, 1:>65, 1:>70, 1:>75, 1:>80, 1:>85, 1:>90, 1:>95, 1:>100, 1:>110, 1:>120, 1:>130, 1:>140, 1:150, 1:>160, 1:>170, 1:>180, 1:>190, 1:>200, 1:>220, 1:>240, 1:>260, 1:>280, 1:>300, 1:>320, 1:>340, 1:>360, 1:>380, 1:>400, 1:>420, 1:>440, 1:>460, 1:>480, or about 1:>500 or more. Preferred ratios include 1:>10.

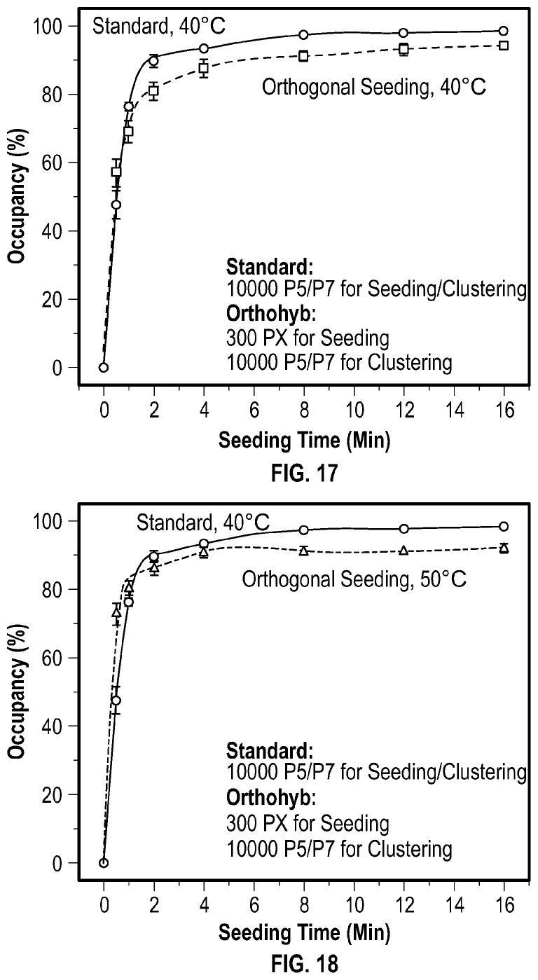

The effect of temperature on seeding time for orthogonal seeding strategies was considered and is shown in Example 5 and FIGS. 17-18. It can be seen that orthogonal seeding strategies achieve similar levels of occupancy versus standard protocols. Increasing the temperature enables faster seeding.

In an embodiment seeding is carried out at a temperature of around 40° C. to 60° C., preferably 40° C. or 50° C.

In an embodiment seeding is carried out for at least 1 minutes at 50° C., preferably for between 5 and 10 minutes.

In an embodiment, the library is seeded at a concentration of between 50-2000 pM. In an embodiment, the library is seeded at a concentration of 300 pM.

In an embodiment clustering is carried out at a temperature of between 35° C. and 60° C. In an embodiment clustering is carried out at a temperature of about 38° C.

In an embodiment clustering is carried out for at least 10 minutes, preferably at least 30 minutes

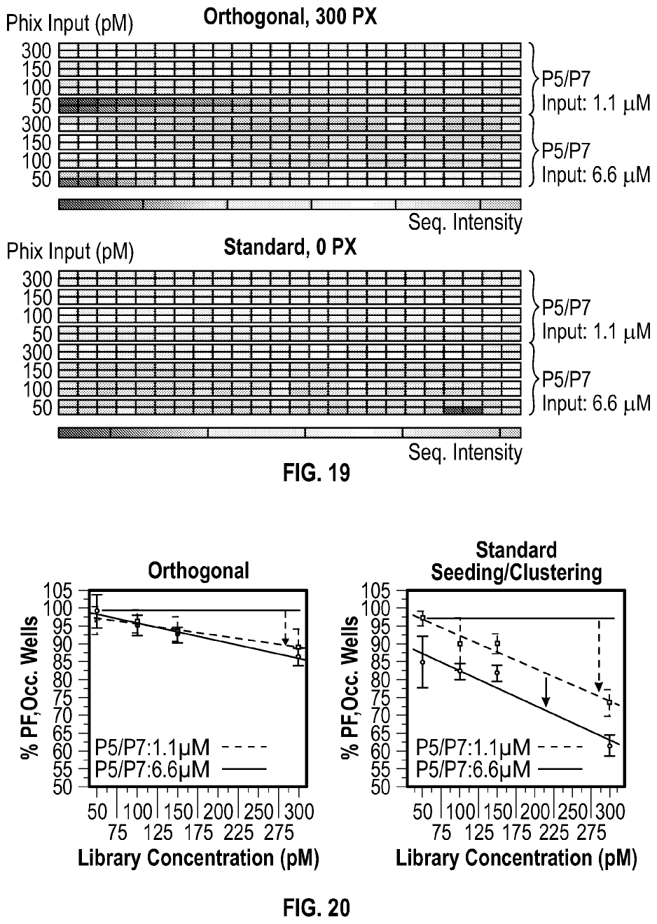

FIGS. 19-22 demonstrate that orthogonal seeding strategies according to the present disclosure enable high signal intensity without a corresponding reduction in % PF (due to polyclonality). There is a marked contrast from standard seeding/clustering (where results are heavily influenced by varying both library concentration and/or primer density) and the present disclosure where varying these parameters largely does not impact results. As such, the present disclosure allows for optimisation of both library concentration and cluster density to suit the particular needs of the analysis.

Furthermore, there was a marked reduction in error rates using orthogonal seeding strategies according to the present disclosure. Overall errors were ˜1% after 150 cycles versus nearly 8% in some circumstances under standard seeding/clustering. Error rate is linked to signal-to-noise ratio, which means that decoupled strategies according to the present disclosure have better signal-to-noise ratios. This may be due to increased clonality (i.e. a reduction in polyclonality) within clusters. A better signal-to-noise ratio can advantageously allow for longer runs. As such, the present disclosure allows for an increase in the number of runs whilst maintaining low error rates.

In an embodiment of the present disclosure, after 150 cycles the mean error rate is less than 1.5%, preferably less than 1.2% preferably less than 1%, preferably less than 0.5%, preferably less than 0.25%, preferably less than 0.1%, preferably 0.05%. In a further embodiment, after 200 cycles the mean error rate is less than 2%, preferably less than 1.5%. preferably less than 1%, preferably less than 0.5% preferably less than 0.25%, preferably less than 0.1%, preferably 0.05%. In a further embodiment, after 250 cycles the mean error rate is less than 2.5%, preferably less than 2%, preferably less than 1.5%, preferably less than 1%, preferably less than 0.25%, preferably less than 0.1%. In a further embodiment, after 300 cycles the mean error rate is less than 3%, preferably less than 2.5%, preferably less than 2%, preferably less than 1.5%, preferably less than 1%, preferably less than 0.5%, preferably less than 0.25%, preferably less than 0.1%.

A further application of the orthogonal seeding strategy of the present disclosure is described with reference to FIGS. 23A-23B. Alternative flowcell designs can avoid the need for paired end turn by using two pads containing their own set of unique primers and complementary linearization chemistry (one set for read 1 and one set for read 2). One challenge associated with this configuration is the inability to prevent multiple seeding events from occurring on both PAZAM pads, which generates false paired reads. This can be seen with FIG. 23A. If a single template seeds onto the dual pad surface then clustering will lead to monoclonality. However, if two templates seed onto the pad then it is possible that this can still lead to a true paired read (the first 2-seed example), but in general it is likely that their will either be no paired read or a false paired read (the second and third 2-seed example). The application of orthogonal seeding can minimise or overcome this problem by providing capture agents on only one of the pads. By way of example, this can be achieved through selective surface chemistry on one pad only. Such an approach can eliminate false paired reads by directing template seeding exclusively to the pad displaying the capture motifs. This is shown in FIG. 23B. For the avoidance of doubt, while the Figure is shown with a PCR-library and a PX nucleotide seeding motif, the same principle can be applied more broadly to any library or capture agent (e.g. non-nucleotide binding template seeding).

In a yet further example of the application of the present disclosure, orthogonal seeding strategies can be used in alternative clustering methodologies that take clustering off the flowcell and instead copy libraries onto designed particles. The current approach whereby multiplexed samples are copied in one pot on the flowcell leads to possible cross-contamination. Taking clustering off the flowcell can remove index hopping, since samples can be clustered independently and subsequently mixed prior to flowcell loading. Off flowcell clustering also may simplify flowcell architecture design. The present disclosure allows particles to have a single point for library attachment thereby enabling the generation of monoclonal clustered particles. For example, the present disclosure enables monoclonal attachment by providing a hybridization sequence that is unique from clustering oligos. Other, non-nucleotide approaches are equally possible.

### EXAMPLES

### Example 1

The robustness of a ds-library according to the present disclosure versus a ss-library was evaluated. The results are shown in FIG. 11 In which the graph compares 1^{st} base intensity obtained after different staging time at 35° C. The library is diluted in hybridization buffer (HT1) and incubated for a specified period of between 0 and 180 minutes at 35° C. before being introduced in the flow cell for hybridization with the seeding primers. If the library is single stranded, it slowly rehybridize with itself, which prevents seeding from happening and leads to decreasing occupancy and sequencing intensity. Alternatively, with double stranded PX-libraries where the PX is always available, no rehybridization occurs and, consequently, there is no decrease in intensity.

### Example 2

The ability of PX-assisted ds-library seeding was evaluated as set out schematically in FIGS. 13A-13C. In FIG. 13A 300 pM of PX-libraries was introduced into a standard flow cells after denaturation (P5/P7 grafting: 1.1 M. PX grafting: 0 pM). Since it's denatured and the P5/P7 on the libraries are available for seeding, clustering intensity could then be detected. In FIG. 13B 300 pM of PX-libraries was introduced into a standard flow cells without denaturation (P5/P7 grafting: 1.1 pM. PX grafting: 0 μM). Since it's not denatured and the P5/P7 on the libraries are not available for seeding, clustering intensity could not be detected. In FIG. 13C 300 pM of PX-libraries was introduced into an orthogonal hybridisation flow cells without denaturation (P5/P7 grafting: 1.1 μM. PX grafting: 0.07 pM). Although it's not denatured, the presence of FC-PX allows the libraries to seed and cluster and clustering intensity can be detected. The clustering intensity results are shown in FIG. 12.

### Example 3

The relationship between capture agent surface density and library concentration was evaluated by seeding different sets of conditions with 300 pM of PX-libraries and clustering for 60 minutes to measure occupancy, intensity and % PF. PX input concentrations were titrated from 0.3 nM to 0.3 pM, while P5/P7 grafting input remained constant at 1.1 μM. As measured by the fluorescence de-hybridization assay, the PX input titration resulted in the number of PX motifs varying from an average of ˜2 to 2500 strands per well, while P5/P7 input led to ˜10000 P5/P7 per nanowell.

It can be seen from FIG. 15 that the optimal number of PX per nanowell decreases with an increase in library concentration. For instance, for 75 pM seeding concentration, 625 PX per nanowell led to the maximum % PF and C1 intensity, while seeding at 300 pM required only 39 PX per nanowell to reach similar performance. The low number of PX motifs necessary to maximize % PF with orthogonal hybridization demonstrates a more efficient seeding process using the orthogonal seeding strategies of the present disclosure.

### Example 4

An experiment was conducted to demonstrate that orthogonal seeding can allow improvements in sequencing intensity by working at higher clustering primer (P5/P7) grafting densities without affecting clonality. A P5/P7 grafting titration was performed from 0.37 to 9.9 pM, while co-grafting each lane with 5 nM of PX (˜39 pX per nanowell). By maintaining the number of PX per nanowell constant, FIG. 16 shows that C1 intensity is increased by increasing P5/P7 density without dramatically impacting clonality (% PF>70% at 9.9 pM P5/P7).

### Example 5

The effect of temperature on seeding time for orthogonal seeding strategies was evaluated by introducing 300 pM of PX-library to the flow cell and incubating for various amounts of time before washing any unbound libraries from the flow cell. The occupancy for a given incubation time was then measured. The results are shown in FIGS. 17-18.

In a first experiment carried out at 40° C. for both the standard primer approach (10,000 P5/P7 for seeding/clustering) and the orthogonal approach (300 PX for seeding/10,000 P5/P7 for clustering. P5/P7 input: 1.1 μM; PX: 0, 0.025 μM; Library: PhiX; Concentration: 300 pM; ExAmp: RAS6T; Measurement: Occupancy obtained with Scope3 after 1st base incorporation. With the orthogonal seeding approach, 300 PX per nanowell to perform seeding resulted in similarly high levels of occupancy after short seeding time.

The experiment was repeated but the temperature for the orthogonal seeding approach was increased to 50° C. P5/P7 input: 1.1 pM; PX: 0, 0.025 pM; Library: PhiX; Concentration: 300 pM; ExAmp: RAS6T; Measurement: Occupancy obtained with Scope3 after 1st base incorporation. It was shown that the rate of hybridization was boosted at higher temperature, reaching maximum occupancy faster.

### Example 6

A further experiment was conducted to demonstrate improvements in cluster signal intensity versus clonality. A flowcell utilising an orthogonal seeding strategy was compared against a standard seeding and clustering protocol. Two different P5/P7 grafting concentrations were assessed, 1.1 μM and a higher 6.6 μM. The higher concentration under standard conditions would be expected to boost the signal intensity but create clonality issues. FIG. 19 shows the signal intensity of the system.

FIG. 20 shows % PF of occupied wells when both the library and primer input concentration is varied. This measure provides the best representation of clonality. It can be seen that with the decoupled seeding approach of the present disclosure there is no significant difference in clonality as the clustering primers are increased. Said another way, clustering primer surface density (e.g. P5/P7 surface density) does not affect clonality with decoupled seeding strategies according to the present disclosure. In contrast, under a standard seeding/clustering protocol, there is a clear decrease in % PF both when the primer density is increased and whether library concentration is increased.

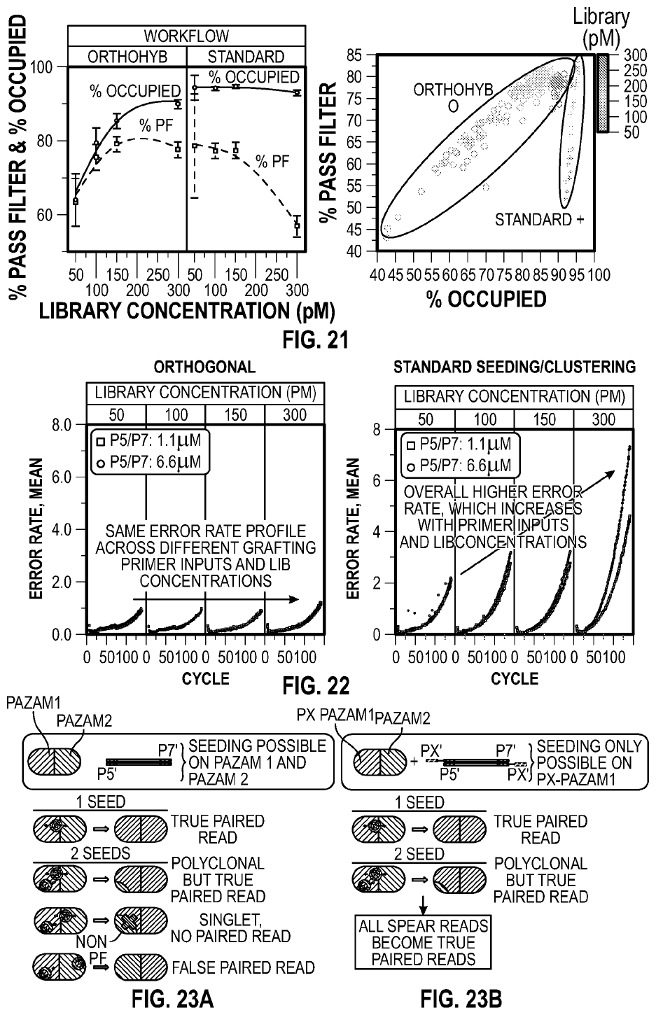

FIG. 21 focuses on higher primer density with a 6.6 pM input and shows % occupied and global % PF vs library concentrations. It can be seen that using an orthogonal seeding strategy having a low number of capture (PX) sites per nanowell was able to minimize the number of strands captured in the nanowells. It can be seen at low library concentrations, there was almost 100% clonality. As library concentrations are increased the rate of occupation and consequently overall % PF increased.

In contrast, using a non-orthogonal standard seeding/clustering approach with high primer density of 6.6 μM, occupancy is saturated across all concentrations. The number of primers on the flowcell means that occupancy does not even scale with library concentration. However, as library concentration increases % PF decreases. This is due to increasing polyclonality within nanowells.

FIG. 22 investigates error rate using decoupled strategies according to the present disclosure. With a decoupled seeding approach, there is no difference in error rate at low or higher clustering primer density. There is also no change in error rate due to increasing library concentration. After 150 cycles, overall errors are limited to a maximum of ˜1%. In contrast, under a standard approach, there are higher error rates, which increase both with increased primer density and library concentration. Error rates approach 8% at high library concentrations and high primer densities.

Further data was also generated comparing orthogonal versus standard seeding/cluster generation. These are shown in tables 1 and 2 below and demonstrate the improved clonality seen with orthogonal hybridisation which results in better signal-to-noise ratio, for the dominant cluster within the nanowell, and in turn better base-calling ability (lower error rates) and better resistance to phasing/prephasing.

TABLE 1
orthogonal, 300PX

| PhiX | | | | | | | | | | |
|---|---|---|---|---|---|---|---|---|---|---|
| input | Pha [symbol] / | % >= | % >= Q30 | Error Rate | Error Rate | Error Rate | Error Rate | Intensity | | |
| (pM) | Preph [symbol] | Q30 | (Last 10 Cycles) | (%) | [symbol] cycle (%) | 75 cycle (%) | 100 cycle (%) | Cycle 1 | | |
| 300 | [symbol] | [symbol] | [symbol] | [symbol] | [symbol] | [symbol] | [symbol] | [symbol] | | |
| 150 | [symbol] | [symbol] | [symbol] | [symbol] | [symbol] | [symbol] | [symbol] | [symbol] | | |
| 100 | [symbol] | [symbol] | [symbol] | [symbol] | [symbol] | [symbol] | [symbol] | [symbol] | {close oversize brace} | P5/P7 input: 1.1 μM |
| 50 | [symbol] | [symbol] | [symbol] | [symbol] | [symbol] | [symbol] | [symbol] | [symbol] | | |
| 300 | [symbol] | [symbol] | [symbol] | [symbol] | [symbol] | [symbol] | [symbol] | [symbol] | | |
| 150 | [symbol] | [symbol] | [symbol] | [symbol] | [symbol] | [symbol] | [symbol] | [symbol] | | |
| 100 | [symbol] | [symbol] | [symbol] | [symbol] | [symbol] | [symbol] | [symbol] | [symbol] | {close oversize brace} | P5/P7 input: 6.6 μM |
| 50 | [symbol] | [symbol] | [symbol] | [symbol] | [symbol] | [symbol] | [symbol] | [symbol] | | |

[symbol]

indicates data missing or illegible when filed

TABLE 2
standard seeding/clustering

| PhiX | | | | | | | | | | |
|---|---|---|---|---|---|---|---|---|---|---|
| input | Pha [symbol] / | % >= | % >= Q30 | Error Rate | Error Rate | Error Rate | Error Rate | Intensity | | |
| (pM) | Preph [symbol] | Q30 | (Last 10 Cycles) | (%) | [symbol] cycle (%) | 75 cycle (%) | 100 cycle (%) | Cycle 1 | | |
| 300 | [symbol] | [symbol] | [symbol] | [symbol] | [symbol] | [symbol] | [symbol] | [symbol] | | |
| 150 | [symbol] | [symbol] | [symbol] | [symbol] | [symbol] | [symbol] | [symbol] | [symbol] | | |
| 100 | [symbol] | [symbol] | [symbol] | [symbol] | [symbol] | [symbol] | [symbol] | [symbol] | {close oversize brace} | P5/P7 input: 1.1 μM |
| 50 | [symbol] | [symbol] | [symbol] | [symbol] | [symbol] | [symbol] | [symbol] | [symbol] | | |
| 300 | [symbol] | [symbol] | [symbol] | [symbol] | [symbol] | [symbol] | [symbol] | [symbol] | | |
| 150 | [symbol] | [symbol] | [symbol] | [symbol] | [symbol] | [symbol] | [symbol] | [symbol] | | |
| 100 | [symbol] | [symbol] | [symbol] | [symbol] | [symbol] | [symbol] | [symbol] | [symbol] | {close oversize brace} | P5/P7 input: 6.6 μM |
| 50 | [symbol] | [symbol] | [symbol] | [symbol] | [symbol] | [symbol] | [symbol] | [symbol] | | |

[symbol]

indicates data missing or illegible when filed

In conclusion, the present disclosure is directed to the use of orthogonal capture moieties, which decouples template capture from clustering. This decoupling leads to a number of advantages. It is possible to control flowcell design to optimise template capture to ensure clonality, but also to optimise clustering density to maximise signal. This leads to improved % PF due to the ability to maximise the likelihood of only one template seeding each nanowell. This also leads to improved signal intensity due to the ability to maximise clustering primers. In addition, the present disclosure leads to reduced error rates and thereby improved signal to noise ratio. This enables longer runs which in turn provides system advantages. The present disclosure can be seeded as a dsDNA library. This removes the need to denature the library and avoids issues around rehybridisation and library denaturation. Thus, steps are removed in the overall process and reliability can be improved by avoiding the risk of library degradation. The present disclosure also improves the possibility of dual-pad techniques and off-flow cell clustering.

### Sequence Listing

SEQ ID NO: 1: P5 sequence
AATGATACGGCGACCACCGAGATCTACAC
SEQ ID NO: 2: P7 sequence
CAAGCAGAAGACGGCATACGAGAT
P5′ sequence (complementary to P5)
SEQ ID NO: 3
GTGTAGATCTCGGTGGTCGCCGTATCATT
P7′ sequence (complementary to P7)
SEQ ID NO: 4
ATCTCGTATGCCGTCTTCTGCTTG
PX′-P5:
SEQ ID NO: 5
5′ CCTCCTCCTCCTCCTCCTCCTCCT/iSp9/AATGATACGGCGACCA
CCGA 3′
PX′-P7:
SEQ ID NO: 6
5′ CCTCCTCCTCCTCCTCCTCCTCCT/iSp9/CAAGCAGAAGACGGCA
TAC 3′
PX substrate sequence:
SEQ ID NO: 7
5′ AGGAGGAGGAGGAGGAGGAGGAGG/iSp9/U-alkyne 3′
PX
SEQ ID NO: 8
AGGAGGAGGAGGAGGAGGAGGAGG
cPX (PX′)
SEQ ID NO: 9
CCTCCTCCTCCTCCTCCTCCTCCT
PA
SEQ ID NO: 10
GCTGGCACGTCCGAACGCTTCGTTAATCCGTTGAG
cPA (PA′)
SEQ ID NO: 11
CTCAACGGATTAACGAAGCGTTCGGACGTGCCAGC
PB
SEQ ID NO: 12
CGTCGTCTGCCATGGCGCTTCGGTGGATATGAACT
cPB (PB′)
SEQ ID NO: 13
AGTTCATATCCACCGAAGCGCCATGGCAGACGACG
PC
SEQ ID NO: 14
ACGGCCGCTAATATCAACGCGTCGAATCCGCAACT
cPV (PC′)
SEQ ID NO: 15
AGTTGCGGATTCGACGCGTTGATATTAGCGGCCGT
PD
SEQ ID NO: 16
GCCGCGTTACGTTAGCCGGACTATTCGATGCAGC
cPD (PD′)
SEQ ID NO: 17
GCTGCATCGAATAGTCCGGCTAACGTAACGCGGC

What is claimed is:

1. A solid support for use in sequencing comprising a plurality of capture moieties adapted to capture a template and a plurality of clustering primers; wherein the capture moieties are orthogonal to the clustering primers.

2. The solid support according to claim 1, wherein the ratio of capture moieties clustering primers is about 1:2, 1:3, 1:4, 1:5, 1:6, 1:7, 1:8, 1:9, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, 1:110, 1:120, 1:130, 1:140, 1:150, 1:160, 1:170, 1:180, 1:190, 1:200, 1:220, 1:240, 1:260, 1:280, 1:300, 1:320, 1:340, 1:360, 1:380, 1:400, 1:420, 1:440, 1:460, 1:480, or about 1:500 or more; or wherein the ratio of capture moieties: clustering primer is about 1:>2, 1:>3, 1:>4, 1:>5, 1:>6, 1:>7, 1:>8, 1:>9, 1:>10, 1:>15, 1:>20, 1:>25, 1:>30, 1:>35, 1:>40, 1:>45, 1:>50, 1:>55, 1:>60, 1:>65, 1:>70, 1:>75, 1:>80, 1:>85, 1:>90, 1:>95, 1:>100, 1:>110, 1:>120, 1:>130, 1:>140, 1:150, 1:>160, 1:>170, 1:>180, 1:>190, 1:>200, 1:>220, 1:>240, 1:>260, 1:>280, 1:>300, 1:>320, 1:>340, 1:>360, 1:>380, 1:>400, 1:>420, 1:>440, 1:>460, 1:>480, or about 1>500 or more.

3. The solid support according to any preceding claim wherein, the clustering primers comprise P5 and P7 primers; optionally wherein the P5 primer comprises a sequence comprising SEQ ID NO: 1 or variant thereof, and/or wherein the P7 primer comprises a sequence comprising SEQ ID NO: 3 or variant thereof.

4. The solid support according to any preceding claim wherein the capture moiety comprises an oligonucleotide seeding sequence.

5. The solid support according to any preceding claim wherein the oligonucleotide seeding sequence comprises between 10 and 30 nucleotides, or between 20 and 30 nucleotides.

6. The solid support according to claim 5, wherein the oligonucleotide seeding sequence comprises a sequence comprising SEQ ID NO: 7 or a variant thereof, or includes a sequence comprising SEQ ID NO: 8 or a variant thereof, SEQ ID NO: 10 or a variant thereof, SEQ ID NO: 12 or a variant thereof, SEQ ID NO: 14 or a variant thereof, or SEQ ID NO: 16 or a variant thereof.

7. The solid support according to any one of claims 1 to 3, wherein the capture moiety is non-nucleotide and the capture moiety binds to a template by non-covalent interactions or by covalent interactions.

8. The solid support according to claim 6, wherein the capture moiety binds to a template by non-covalent interactions (including molecular recognition, such as ionic bonding, hydrogen bonding, hydrophobic interactions, van der Waals interactions and/or 7n-7n interactions) and/or host-guest interactions (including interactions formed between cucubiturils with adamantanes (e.g. 1-adamantylamine), ammonium ions (e.g. amino acids), ferrocenes; cyclodextrins with adamantanes (e.g. 1-adamantylamine), ammonium ions (e.g. amino acids), ferrocenes, calixarenes with adamantanes (e.g. 1-adamantylamine), ammonium ions (e.g. amino acids), ferrocenes; crown ethers (e.g. 18-crown-6, 15-crown-5, 12-crown-4) or cryptands (e.g. [2.2.2]cryptand) with cations (e.g. metal cations, ammonium ions); avidins (e.g. streptavidin) and biotin; and antibodies and haptens).

9. The solid support according to claim 8, wherein the capture moiety binds to a template via an avidin (e.g. streptavidin) and biotin interaction.

10. The solid support according to any one of claim 7, wherein the capture moiety binds to a template by covalent interactions, wherein the covalent interaction may be reversible or non-reversible.

11. The solid support according to claim 10, wherein the covalent interaction is selected from alkylene linkages; alkenylene linkages; alkynylene linkages; ether linkages, such as ethylene glycol, propylene glycol, polyethylene glycol; amine linkages; ester linkages; amide linkages; carbocyclic or heterocyclic linkages; sulfur-based linkages, such as thioether, disulfide, polysulfide, or sulfoxide linkages; acetals; hemiaminal ethers; aminals; imines; hydrazones; boron-based linkages, such as boronic and borinic acids/esters; silicon-based linkages, such as silyl ether, siloxane; and phosphorus-based linkages, such as phosphite, phosphate.

12. The solid support according to any preceding claim, wherein the solid support is a flowcell, and wherein the flowcell comprises a plurality of nanowells.

13. The solid support according to any one of claims 1 to 11, wherein the solid support is a microbead.

14. The solid support according to claim 12 or claim 13, wherein, on average, each nanowell or microbead comprises between around 1 to 5000 capture moieties; preferably between around 1 to 2500, 1 to 1000, 1 to 625, 1 to 500, 1 to 300, 1 to 200, 1 to 156, 1 to 100, 1 to 80, 10 to 80, 1 to 60, 20 to 60, 30 to 50, 1 to 50, or around 35 to 45, or around 35, 36, 37, 38, 39, 40, 41, 42, 43, 43 or 45 capture moieties.

15. The solid support according to claim 12 or 13, wherein on average each nanowell or microbead comprises above around 5000 clustering primers; preferably above 6000, 7000, 8000, 9000, 10,000, 11,000, 12,000, 13,000, 14,000, 15,000, 16,000, 17,000, 18,000, 19,000 or above 20,000 clustering primers.

16. A nucleotide template library comprising a plurality of templates, wherein the templates comprise an insert and adaptor regions; wherein each adaptor region comprises a clustering primer and a complementary capture moiety, wherein the clustering primer and complementary capture moiety are orthogonal.

17. The nucleotide template library according to claim 16, wherein a spacer region is provided between the clustering primer and the complementary capture moiety.

18. The nucleotide template library according to claim 17, wherein the spacer region is a linker, wherein the linker may optionally be a PEG linker.

19. The nucleotide template library according to any one of claims 16 to 18, wherein the template comprises P5′ and P7′ primers; optionally wherein the P5′ primer comprises a sequence comprising SEQ ID NO: 2 or variant thereof, and/or wherein the P7′ primer comprises a sequence comprising SEQ ID NO: 4 or variant thereof.

20. The nucleotide template library according to any one of claims 16 to 19, wherein the complementary capture moiety is complementary to the capture moiety as defined in any one of claims 4 to 11.

21. The nucleotide template library according to any one of claims 16 to 20, wherein the library is a double stranded library.

22. The nucleotide template library according to any one of claims 16 to 21, wherein the template further comprises an index sequence (e.g. i5), a first sequencing binding site (e.g. SBS3), a second sequencing binding site (e.g. SBS12), and/or a second index sequence (e.g. i7); wherein if the template is a double stranded template the complementary sequences are also provided.

23. An orthogonal capture fragment, comprising:
a first primer binding sequence substantially complementary to a primer binding sequence on a template (optionally wherein the first primer binding sequence is SEQ ID NO: 1 or variant thereof; or SEQ ID NO: 3 or variant thereof);
a complementary capture moiety, wherein said complementary capture moiety may optionally be complementary to the capture moiety as defined in any one of claims 4 to 11; and
a linker between said first primer binding sequence and said complementary capture moiety, wherein the linker may optionally be a PEG linker;
wherein said complementary capture moiety is orthogonal to said first primer binding sequence.

24. A method of sequencing a target nucleotide, wherein said method includes the step of preparing a double stranded library comprising templates as defined in any one of claims 16 to 23.

25. The method of claim 24, wherein the double stranded library is applied to a solid support surface according to any one of claims 1 to 15; wherein said complementary capture moiety on said templates are captured by said capture moieties on said support surface, such that the template library is seeded.
